(19) United States
(12) Patent Application Publication
(10) Pub. No.: US 2020/0375965 A1
(43) Pub. Date: Dec. 3, 2020

(54) Targeting Lipid Metabolism and Free Fatty Acid (FFA) Oxidation to Treat Diseases Mediated by Resident Memory T Cells (TRM)

(71) Applicant: The Brigham and Women's Hospital, Inc., Boston, MA (US)

(72) Inventors: Thomas S. Kupper, Belmont, MA (US); Rachael Clark, Belmont, MA (US); Youdong Pan, Jamaica Plain, MA (US)

(21) Appl. No.: 16/970,879

(22) PCT Filed: Feb. 15, 2019
(86) PCT No.: PCT/US2019/018341

§ 371 Date: Aug. 18, 2020

Related U.S. Application Data
(60) Provisional application No. 62/632,067, filed on Feb. 19, 2018.

(51) Int. Cl. A61K 31/4458; A61K 31/17; A61K 31/167; A61K 31/216; A61K 31/336

(52) U.S. Cl. CPC A61K 31/4458; A61K 31/17; A61K 45/06; A61K 31/216; A61K 31/336; A61K 31/167

(57) ABSTRACT

Methods for treating, or reducing risk of development or progression of, a tissue-resident memory T cells (TRM)-mediated disease, comprising administering a therapeutically effective amount of one or more inhibitors of exogenous lipid and free fatty acid uptake or of mitochondrial beta oxidation of internalized exogenous FFA (e.g., inhibitors of CD36 and/or FABP antagonists, e.g., inhibitors of FABP4 and/or FABP5, and/or CPT1) to a subject in need thereof.

### CLAIM OF PRIORITY

This application claims the benefit of U.S. Provisional Patent Application Ser. No. 62/632,067, filed on Feb. 18, 2018. The entire contents of the foregoing are hereby incorporated by reference.

### FEDERALLY SPONSORED RESEARCH OR DEVELOPMENT

This invention was made with government support under Grant Nos AI041707, AI127654, AI097128, and AR063962 awarded by the National Institutes of Health. The government has certain rights in the invention.

### BACKGROUND

Memory T cells protect the host through rapid recall responses to pathogens. A population of memory T cells vital for host defense, tissue-resident memory T cells (T_{RM}), has recently been characterized¹⁻⁴. T_{RM} reside in epithelial barrier tissues and persist for long periods of time at the interface between host and environment^{3,4}. Upon re-infection, CD8⁺ T_{RM} provide a rapid antigen-specific immune response, creating an inflammatory and antiviral microenvironment that facilitates pathogen elimination⁶⁻⁹.

### SUMMARY

The inventors have discovered the therapeutic use of inhibitors of exogenous lipid and free fatty acid (FFA) uptake (e.g., inhibitors of CD36 and inhibitors of Fatty Acid Binding Proteins (FABPs) such as FABP4 and FABP5) and/or inhibitors of motichondrial beta-oxidation of FFAs, to treat diseases mediated by Resident Memory T Cells (TRM). TRM-mediated diseases include many autoimmune and autoinflammatory diseases and disorders, which include diseases of skin (e.g., psoriasis, vitiligo, graft vs host disease, CTCL, contact dermatitis, alopecia areata, eczematous dermatitis), GI tract (e.g., Crohns disease, ulcerative colitis), lung (e.g., asthma), joint (e.g., rheumatoid arthritis, spondyloarthropathies), CNS (e.g., multiple sclerosis), and endocrine system (e.g., Type I diabetes), and more.

Thus provided herein are methods for treating, or reducing risk of development or progression of, a TRM-mediated disease, comprising administering a therapeutically effective amount of one or more inhibitors of exogenous lipid and free fatty acid uptake or of mitochondrial beta oxidation of internalized exogenous FFA (e.g., inhibitors of CD36 and/or FABP antagonists, e.g., inhibitors of FABP4 and/or FABP5) to a subject in need thereof. In some embodiments, the TRM-mediated diseases is selected from the group consisting of autoimmune and autoinflammatory diseases and disorders, e.g., diseases of skin (psoriasis, vitiligo, graft vs host disease, CTCL, contact dermatitis, alopecia areata, eczematous dermatitis), GI tract (Crohns disease, ulcerative colitis), lung (asthma), joint (rheumatoid arthritis, spondyloarthropathies), CNS (multiple sclerosis), and endocrine system (Type I diabetes).

Thus, provided herein are methods for treating or reducing risk of development or progression of an immune or inflammatory disease. The methods include administering a therapeutically effective amount of (i) one or more inhibitors of mitochondrial exogenous lipid or free fatty uptake and/or (ii) one or more inhibitors of mitochondrial fatty acid oxidation or metabolism to a subject in need thereof. Also provided herein are compositions comprising (i) one or more inhibitors of mitochondrial exogenous lipid or free fatty uptake and/or (ii) one or more inhibitors of mitochondrial fatty acid oxidation or metabolis, for use in treating or reducing risk of development or progression of an immune or inflammatory disease.

In some embodiments, the disease is mediated by resident memory T cells (TRM). In some embodiments, the TRM-mediated disease is an autoimmune or auto-inflammatory disease or disorder involving non-lymphoid tissue. In some embodiments, the disease is an autoimmune or auto-inflammatory disease or disorder is selected from one or more of the following: (a) diseases of the skin; (b) diseases of the gastrointestinal (GI) Tract; (c) endocrine or metabolic diseases (d) diseases of the lung; (e) diseases of the bones or joints or (f) diseases of the CNS.

In some embodiments, the disease of the skin is selected from (a) psoriasis; (b) vitiligo; (c) graft vs host disease; (d) contact dermatitis; (e) alopecia areata; or (f) eczematous dermatitis.

In some embodiments, the disease of the GI tract is Crohn's Disease, irritable bowel disease, or ulcerative colitis.

In some embodiments, the disease of the lung is asthma.

In some embodiments, the endocrine or metabolic disease is Type I diabetes (insulin dependent diabetes mellitus).

In some embodiments, the disease of the bones or joints is rheumatoid arthritis or a spondylarthropathy.

In some embodiments, the disease of the CNS is multiple sclerosis.

In some embodiments, the one or more inhibitors of mitochondrial exogenous lipid or free fatty uptake comprise an inhibitor or antagonist of CD36 or a Fatty Acid Binding Protein (FABP).

In some embodiments, the inhibitor or antagonist of FABP is an inhibitor or antagonist of FABP4 or FABP5. In some embodiments, the inhibitor or antagonist of FABP4 or FABP5 is carbazole butanoic acid, aryl sulfonamide, sulfonylthiophene derivative, 4-hydroxypyrimidine, tetrahydrocarbazol e derivative, 2,3-dimethylindole derivative, benzoylbenzene, biphenyl-alkanoic acid derivative, 2-oxazole-alkanoic acid derivative, tetrahydropyrimidone, pyridone, pyrazinone, aryl carboxylic acid, tetrazole, triazolopyrimidinone, BMS309403; pyrazole, 4-{[2-(methoxycarbonyl)-5-(2-thienyl)-3-thienyl]amino}-4-oxo-2-butenoic acid or ((2′-(5-ethyl-3,4-diphenyl-1H-pyrazol-1-yl)(1,1′-biphenyl)-3-yl)oxy)-acetic acid; an indole derivative, triazolopyrimidinone derivative, Pyrazole; SBFI26 (alpha-2,4-diphenylcyclobutane-1,3-dicarboxylic acid mono-1-naphthyl ester) and other α-truxillic acid derivatives, e.g., SBFI50 (alpha-2,4-diphenylcyclobutane-1,3-dicarboxylic acid mono-2-naphthyl ester), SBFI60 (alpha-2,4-diphenylcyclobutane-1,3-dicarboxylic acid mono-1-naphthyl amide), and SBFI62 (2,4-diphenylcyclobutane-1,3-dicarboxylic acid di-1-naphthyl amide), or an inhibitory nucleic acid.

In some embodiments, the one or more inhibitors of mitochondrial fatty acid beta oxidation or metabolism is an inhibitor of carnitine palmitoyltransferase 1 (CPT1). In some embodiments, the inhibitor of CPT1 is etomoxir; 2-tetradecylglycidic acid (TDGA); ST1326 (Teglicar); or perhexiline (2-(2,2-dicyclohexylethyl)piperidine) perhexiline (2-(2,2-dicyclohexylethyl)piperidine), or a derivative thereof, or an inhibitory nucleic acid.

In some embodiments, the methods include administering a PPAR gamma inhibitor. In some embodiments, the PPAR gamma inhibitor is GW9662, or a derivative thereof.

In some embodiments, the disease is a disease of the skin, and administration of the one or more inhibitors is by topical delivery.

In some embodiments, the composition is formulated for topical administration.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIGS. 1A-G. Skin CD8⁺ tissue-resident memory T cells (T_{RM}) display increased expression of fatty acid binding protein 4 (FABP4) and FABP5. A, Principal component analysis (PCA) of gene-expression data for CD8⁺ T cell subtypes. Each timepoint represents an individual experiment wherein mRNA was pooled from 15-20 mice from 3-4 independent biological groups (5 mice/group). B, Pearson correlation coefficients among CD8⁺ T cell subtypes. C, Heatmap of differentially expressed genes selected from a pair-wise comparison between OT-I T_{RM} (day 30) and T_{CM}. Genes are shown with color indicating z-score across rows. D, Quantitative real-time PCR (qRT-PCR) analysis of Fabp4 and Fabp5 expression in T_{N}, T_{CM}, T_{EM} and T_{RM} (day 30). E, qRT-PCR analysis of Fabp4 and Fabp5 gene expression in skin CD103⁻ and CD103⁺ T_{RM} (day 30). F, qRT-PCR analysis of Pparγ expression in T_{N}, T_{CM}, T_{EM} and T_{RM} (day 30). G, Effect of lentiviral Pparγ siRNA knockdown on Fabp4 and Fabp5 expression in OT-I CD8⁺ T_{RM}. Graphs in D-G show mean±s. d. from triplicates. β-actin was used as internal control and mRNA was normalized to T_{N} samples (D, E, F) or T_{RM} transduced with a lentiviral vector encoding scrambled siRNA (G). T cells from 15-20 mice were pooled for each group. ns, not significant, **p<0.01.

FIGS. 2A-H. Loss of Fabp4 and Fabp5 decreases fatty acid uptake and metabolism by CD8⁺ T_{RM} and impairs their long-term maintenance. A, Representative histograms and average MFI of Bodipy FL C16 uptake by T_{N}, T_{CM}, T_{EM} or T_{RM} (day 30). Graphs show mean±s. d. of 5 mice. B, Representative histograms and average MFI of Bodipy FL C16 uptake in OT-I WT and Fabp4/5 dKO T_{RM} with or without pre-incubation with unlabelled palmitate. Graphs show mean±s. d. of 5 mice. C, Enumeration of OT-I WT and Fabp4/5 dKO T_{CM} and T_{RM} at different timepoints post infection. Graphs show mean±s. d. of 10 mice. D, IF and quantification of OT-I WT and Fabp4/5 dKO T_{RM} in infected skin sites 45 days post infection. Graphs show mean±s. d. of 50 fields from 5 mice (10 fields per mouse). Scale bar, 50 μm. E, Representative histograms and quantification of annexin V⁺ cells in OT-I WT and Fabp4/5 dKO T_{RM} 45 days post infection. F, Oxygen-consumption rate (OCR) of OT-I T_{CM}, T_{RM} and Fabp4/5 dKO T_{RM} (day 30) under basal conditions and in response to indicated mitochondria inhibitors. Results were normalized to those of control cells in the presence of BSA. Graphs show mean±s. d. of triplicates. G, Effect of lentiviral Cpt1a siRNA knockdown on OT-I CD8⁺ T_{RM} survival in vivo in infected tissue. H, Representative dot plots and enumeration of WT and Fabp4/5 dKO T_{CM} and T_{RM} 45 days post infection. Cells were gated on VACV-specific pentamer⁺ CD8⁺ T cells. Symbols represent individual mice in E, G, H. ns, not significant, *p<0.05, **p<0.01

FIGS. 3A-G. Skin CD8⁺ T_{RM} lacking Fabp4 and Fabp5 fail to protect mice against viral infectious challenge. A, Schematic of experimental design. B, qRT-PCR analysis of skin viral load at six days post re-infection. Un-Immun.=un-immunized; Immun.=immunized. Symbols represent individual mice. C-D, Representative dot plots and quantification of IFN-γ secretion by OT-I WT or Fabp4/5 dKO T_{RM}. Symbols represent individual mice. E, Schematic of experimental design. F-G, Body weight (BW) (F) and survival measurements (G) of WR-VACV challenged mice, with or without FTY720 treatment. Data are representative of three independent experiments (n=10 mice per group). ns, not significant, *p<0.05, **p<0.01.

FIGS. 4A-D. Human skin CD8⁺ T_{RM} demonstrate increased expression of FABP4 and FABP5. A-B, Representative histograms and average MFI of intracellular staining of FABP4 (A) and FABP5 (B) in human PBMC T_{N}, T_{CM}, and T_{EM} compared to facial skin T_{RM}. Graphs show mean±s. d. of 5 human individuals. C, Hematoxylin and eosin (H&E)-staining of a human psoriatic lesion. Scale bar, 100 μm. D, Representative histograms and average MFI of Bodipy FL C16 uptake by human T_{N}, T_{CM}, T_{EM} or T_{RM} subsets. Graphs show mean±s. d. of 5 human individuals. C, n=15 sections from 5 human individuals and one representative site is shown. **p<0.01.

FIGS. 5A-F. CD8⁺ T_{RM} display increased gene expression of Fabp4 and Fabp5. A, Enumeration of OT-I Thy1.1⁺ cells in infected skin at indicated time post infection. Graphs show mean±s. d. of 10 mice per group. B, Venn diagram analysis of genes differentially expressed in pairwise comparisons between OT-I T_{CM}, T_{EM} and T_{RM} (day 30) relative to that of T_{N} (fold change cutoff, ≥2). C, Absolute qRT-PCR analysis of Fabp gene expression in T_{RM} (day 30). ND, not detectable. D, qRT-PCR assessments of Fabp4 and Fabp5 levels in indicated T cell subsets. OT-I Thy1.1⁺ cells were intravenously transferred into Thy1.2⁺ recipient mice 1 day before mice were infected with 2×10⁶ p.f.u. VACV_{OVA} by intra-tracheal infection. At 45 days later, mice were sacrificed. T_{CM} and T_{EM} were sorted from spleen and T_{RM} were sorted from lung. E, qRT-PCR analysis of Pparγ knockdown efficiency by lentiviral vector encoding two specific and one scrambled siRNA in OT-I CD8⁺ T_{RM}. F, qRT-PCR analysis of Fabp4 and Fabp5 gene expression in OT-I CD8⁺ T_{RM} from mice treated with or without GW9662. Graphs in C, D, E, F show mean±s. d. from triplicates. β-actin was used as internal control and mRNA was normalized to T_{N} samples (D) or T_{RM} with scrambled siRNA transduction (E) or without GW9662 treatment (F). T cells from 15-20 mice were pooled for each sample. ns, not significant, **p<0.01.

FIGS. 6A-D. Relative contribution of FABP4 and FABP5 in fatty acid acquisition and long-term maintenance of skin CD8⁺ T_{RM}. A, Average MFI of Bodipy FL C16 uptake by OT-I WT, Fabp4^{+/-}Fabp5^{+/-}, Fabp4^{-/-} and Fabp5^{-/-} T_{RM} Graphs show mean±s. d. of 5 mice per group. B-D, Enumeration of OT-I WT, Fabp4^{+/-}Fabp5^{+/-}, Fabp4^{-/-} and Fabp5^{-/-} T_{RM} at different timepoints post VACV_{OVA} infection. Thy1.2⁺ CD45.2⁺ recipient mice were given a 1:1 ratio of naive OT-I Thy1.1⁺ CD45.1⁺ WT and OT-I Thy1.1⁺ CD45.2⁺ Fabp4/5 knockout (Fabp4^{+/-}Fabp5^{+/-}, Fabp4^{-/-} or Fabp5^{-/-}) cells one day before infection with 2×10⁶ p.f.u. VACV_{OVA} by skin scarification. Relative percentages of the two T cell populations were analyzed longitudinally by flow cytometry. Graphs show mean±s. d. of 5 mice per group. ns, not significant.

FIGS. 7A-C. OT-I Fabp4/5 dKO effector T cells (T_{eff}) display similar proliferative capacity and tissue-homing receptor expression to WT counterparts. A, Quantification of Ki67⁺ cells in OT-I T_{N}, T_{eff}, T_{CM}, T_{EM} and T_{RM}. Graphs show mean±s. d. of 5 mice per group. B, Average MFI of Bodipy FL C16 uptake by OT-I T_{N}, T_{eff}, T_{CM}, T_{EM} or T_{RM}. Graphs show mean±s. d. of 5 mice per group. *p<0.05, **p<0.01. C, Flow cytometric analysis of T cell proliferation and homing receptor expression on OT-I WT and Fabp4/5 dKO T cells. Thy1.2⁺ CD45.2⁺ recipient mice were given a 1:1 ratio of CFSE-labeled naive OT-I Thy1.1⁺ CD45.1⁺ WT and OT-I Thy1.1⁺ CD45.2⁺ Fabp4/5 dKO cells one day before mice were infected with 2×10⁶ p.f.u. VACV_{OVA} by skin scarification. At 60 hr after infection, proliferation and tissue-homing receptor expression of OT-I cells isolated from draining lymph nodes were analyzed by flow cytometry. Data are representative of three independent experiments (n=5 mice per group). ESL, E-selectin ligand; PSL, P-selectin ligand.

FIG. 8. OT-I Fabp4/5 dKO T_{RM} show a similar surface protein expression phenotype to WT counterparts. Thy1.2⁺ CD45.2⁺ recipient mice were given a 1:1 ratio of naive OT-I Thy1.1⁺ CD45.1⁺ WT and OT-I Thy1.1⁺ CD45.2⁺ Fabp4/5 dKO cells one day before mice were infected with 2×10⁶ p.f.u. VACV_{OVA} by skin scarification. At 45 days post infection, infected skin sites were harvested and phenotype of T_{RM} was examined by flow cytometry. Data are representative of three independent experiments (n=5 mice per group). ESL, E-selectin ligand.

FIGS. 9A-B. Effect of Pparγ lentiviral siRNA knockdown or PPARγ inhibition on CD8⁺ T_{RM} maintenance in peripheral tissue. A, Enumeration of OT-I CD8⁺ T_{RM} transduced with scrambled siRNA or siRNA targeting Pparγ. Pparγ siRNA transduced OT-I cells (together with the same number of congenically scrambled siRNA transduced OT-I cells) were cotransferred into recipient mice that were previously infected with VACV_{OVA} by skin scarification. At 40 days later, mice were sacrificed and the number of siRNA transduced OT-I cells in the infected skin tissue were harvested and enumerated by flow cytometry based on the GFP marker. B, Enumeration of OT-I CD8⁺ T_{RM} in infected skin from mice treated with or without GW9662. Thy1.2⁺ CD45.2⁺ recipient mice were infected with 2×10⁶ p.f.u. VACV_{OVA} by skin scarification. 40 days later, mice were treated with GW9662 daily by intradermal injection for 5 days, after which mice were sacrificed and T_{RM} were enumerated and analyzed by flow cytometry. Graphs show mean±s. d. of 5 mice per group. **p<0.01.

FIGS. 10A-B. Gene expression profile of OT-I Fabp4/5 dKO T_{RM}. A, Principal component analysis (PCA) of gene-expression data for skin infiltrating T cells isolated at day 10 and day 30 post infection. B, Differentially expressed genes selected from a pairwise comparison between OT-I Fabp4/5 dKO T_{RM} and OT-I WT T_{RM}. Thy1.2⁺ CD45.2⁺ recipient mice were given a 1:1 ratio of naive OT-I Thy1.1⁺ CD45.1⁺ WT and OT-I Thy1.1⁺ CD45.2⁺ Fabp4/5 dKO cells one day before mice were infected with 2×10⁶ p.f.u. VACV_{OVA} by skin scarification. At 30 days post infection, OT-I CD8⁺ T_{RM} were sorted from infected skin sites for gene microarray. C, qRT-PCR analysis of genes involved in anti-inflammatory responses (1110 and Socs2), cell apoptosis (Caspase3 and Bcl2113) as well as immune responses (Ccr8, Ccl21, Il1r1, Il6, Cxcl13, Cxcl12, Ccl12, Gzmc, Gzma) in OT-I Fabp4/5 dKO T_{RM} compared to WT T_{RM}. Graphs show mean±s. d. from triplicates. β-actin was used as internal control and mRNA was normalized to OT-I WT T_{RM}. mRNA was pooled from 15 mice from 3 independent biological groups (5 mice/group). **p<0.01.

FIGS. 11A-D. Effect of Cpt1a lentiviral siRNA knockdown or CPT1A inhibition on OT-I CD8⁺ T_{RM} maintenance in peripheral tissue. A, qRT-PCR analysis of Cpt1a lentiviral siRNA knockdown efficiency in OT-I CD8⁺ T_{RM}. B-C, Enumeration of OT-I WT and OT-I Fabp4/5 dKO T_{RM} in infected skin from mice treated with or without etomoxir (B) and trimetazidine (C). Thy1.2⁺ CD45.2⁺ recipient mice were given a 1:1 ratio of naive OT-I Thy1.1⁺ CD45.1⁺ WT and OT-I Thy1.1⁺ CD45.2⁺ Fabp4/5 dKO cells one day before infection with 2×10⁶ p.f.u. VACV_{OVA} by skin scarification. Mice were administered etomoxir or trimetazidine daily by intradermal injection for 5 days from day 40 post infection, after which mice were sacrificed and T_{RM} were analyzed by flow cytometry and enumerated. Symbols represent individual mice. D, Oxygen-consumption rate (OCR) and extracellular acidification rate (ECAR) as measured by the Seahorse assay of skin infiltrating T cells sorted at indicated timepoints post infection under basal conditions. Graphs show mean±s. d. of triplicates. ns, not significant, **p<0.01.

FIGS. 12A-C. Skin CD8⁺ T_{RM} residing in distal infection sites are deficient in long-time survival and virus clearance. A, Ratio of peripheral blood CD3 counts in FTY720 vs untreated mice, illustrating the kinetics of lymphoid sequestration by FTY720. n=5 mice per group. B, Enumeration of OT-I WT and OT-I Fabp4/5 dKO cells infiltrating distal infection sites at different timepoints post VACV_{OVA} infection. Thy1.2⁺ CD45.2⁺ recipient mice were given a 1:1 ratio of naive OT-I Thy1.1⁺ CD45.1⁺ WT and OT-I Thy1.1⁺ CD45.2⁺ Fabp4/5 dKO cells one day before infection with 2×10⁶ p.f.u. VACV_{OVA} by skin scarification at right ears. Relative percentages of the two T cell populations infiltrating left ears were analyzed longitudinally by flow cytometry. Graphs show mean±s. d. of 5 mice per group. C, qRT-PCR analysis of distal infection skin viral load at six days post re-challenge. OT-I WT or OT-I Fabp4/5 dKO cells were adoptively transferred into μMT mice before infection with 2×10⁶ p.f.u. VACV_{OVA} by skin scarification at right ears. 25 days later, mice were re-challenged with VACV_{OVA} at left ear by skin scarification. Un-immun.=unimmunized; Immun.=immunized. Symbols represent individual mice. ns, not significant, **p<0.01.

FIGS. 13A-B. Fatty acid uptake and T cell survival of cd36−/− T_{RM} cells. A, Average MFI of Bodipy FL C16 uptake in OT-I WT and cd36−/− T_{RM} with or without pre-incubation with unlabelled palmitate. Graphs show mean±s. d. of 5 mice. B, Enumeration of OT-I WT and cd36−/− T_{CM} and T_{RM} at different timepoints post infection. Symbols represent individual mice. ns, not significant, **p<0.01.

FIGS. 14A-B. Fatty acid uptake and T cell survival of Fabp4−/−/Fabp5−/− cd36−/− TRM cells. A, Average MFI of Bodipy FL C16 uptake in OT-I WT, Fabp4−/−/Fabp5−/−, cd36−/− and Fabp4−/−/Fabp5−/− cd36−/−T_{RM} with or without pre-incubation with unlabelled palmitate. Graphs show mean±s. d. of 5 mice. B, Enumeration of OT-I WT, Fabp4−/−/Fabp5−/−, cd36−/− and Fabp4−/−/Fabp5−/−cd36−/− T_{CM} and T_{RM} at different timepoints post infection. Symbols represent individual mice. ns, not significant, *p<0.05, **p<0.01.

FIG. 15. TRM depend on exogenous FFA for survival and function, and do not persist in tissues if they cannot utilize this energy source. Effect of FAO inhibition on contact hypersensitivity (CHS) reaction post DNFB re-exposure. DNFB: 20 ul, 0.25% in acetone:olive oil (aOO) (3:1 v/v); Etomoxir (1 ug/site); Trimetazidine (10 ug/site). N=10 mice per group.

### DETAILED DESCRIPTION

Tissue-resident memory T cells (TRM) persist indefinitely in epithelial barrier tissues and protect the host against pathogens¹⁻⁴. However, the biological pathways that enable the long-term survival of T_{RM} are obscure^{4,5}. Here we show that CD8+ TRM generated by viral infection of skin differentially express high levels of several molecules mediating lipid uptake and intracellular transport, including fatty acid binding proteins 4 and 5 (FABP4 and FABP5). We further show that T cell-specific deficiency of Fabp4 and Fabp5 impairs exogenous free fatty acid (FFA) uptake by CD8+ TRM and greatly reduces their long-term survival in vivo, while having no effect on TCM survival in lymph nodes. In vitro, CD8+ TRM but not CD8+ TCM demonstrated increased mitochondrial oxidative metabolism in the presence of exogenous FFA; this increase was not seen in Fabp4/5 dKO CD8+ TRM. Persistence of CD8+ TRM in skin was strongly diminished by inhibition of mitochondrial FFA β-oxidation in vivo. Moreover, skin CD8+ TRM lacking Fabp4/5 were less effective at protecting mice from cutaneous viral infection, and lung Fabp4/5 dKO CD8+ TRM generated by skin VACV infection were less effective at protecting mice from a lethal pulmonary challenge with VACV. Consistent with the mouse data, increased FABP4 and FABP5 expression and enhanced extracellular FFA uptake were also demonstrated in human skin CD8+ TRM in normal and psoriatic skin. These results suggest a critical role of FABP4 and FABP5 in CD8+ TRM maintenance, longevity, and function, and suggest that CD8+ TRM utilize exogenous FFA and their oxidative metabolism to persist in tissue and mediate protective immunity.

Growing evidence suggests that pathogenic TRM mediate tissue-specific immune-mediated diseases as diverse as psoriasis and vitiligo, asthma, inflammatory bowel disease, rheumatoid and spondylo-arthritis, and insulin-dependent diabetes (see, e.g., Park and Kupper, Nat Med. 2015 July; 21(7):688-97). It was hypothesized that the difficulty in achieving durable remission in these diseases is because the genetic program of TRMs is focused on maintaining their indefinite survival in tissues. While TRMs' disease-causing activity can be transiently blocked with immune suppressive drugs, there is currently no means of dislodging these pathogenic cells from tissue. As a result, these diseases are chronic and relapsing. As shown in Example 1 herein, virally-induced CD8+ TRM in skin depended upon uptake of exogenous free fatty acids (FFA), which they used for mitochondrial β oxidation and ATP generation. If either free fatty acid uptake or mitochondrial β oxidation were blocked, CD8+ TRM did not survive in peripheral tissue. Further, as shown herein, blocking TRM lipid uptake and metabolism can be used to dislodge pathogenic TRM from tissue, providing a durable treatment for TRM-mediate immune and inflammatory diseases.

TRM-Mediate Immune and Inflammatory Diseases

The present disclosure provides methods for treating subjects with TRM mediate tissue-specific immune-mediated diseases including (a) diseases of the skin; (b) diseases of the gastrointestinal (GI) Tract; (c) endocrine or metabolic diseases (d) diseases of the lung; (e) diseases of the bones or joints or (f) diseases of the CNS (see, e.g., Park and Kupper, Nat Med. 2015 July; 21(7):688-97). Diseases of the can include (a) psoriasis; (b) vitiligo; (c) graft vs host disease; (d) contact dermatitis; (e) alopecia areata; or (f) eczematous dermatitis. Diseases of the GI tract can include Crohn's Disease, irritable bowel disease, or ulcerative colitis. Diseases of the lung can include asthma. Endocrine or metabolic diseases can include Type I diabetes (insulin dependent diabetes mellitus). Disease of the bones or joints can include rheumatoid arthritis or a spondylarthropathy. Diseases of the CNS can include multiple sclerosis (MS). As used in this context, to “treat” means to ameliorate at least one symptom of the disorder.

In some embodiments, the methods include identifying a subject who has a TRM mediate tissue-specific immune-mediated disease as described herein. Such subjects can be identified by one of skill in the art using known diagnostic methodology.

In some embodiments, a subject treated by a method described herein does not have insulin-dependent diabetes exclude diabetes, psoriasis, or MS.

CD36 Inhibitors

CD36 inhibitors including sulfo-N-succinimidyl oleate (SSO); Ursolic acid; AP5055 or AP5258 (Arteria, Geloen et al., PLoS ONE 7(5): e37633); alvianolic acid B (SAB) SAB or its metabolites, such as rosmarinic acid (RA) and sodium danshensu (DSS); 3-cinnamoyl indole, and 13 pentyl berberine (Xu, Y et al, Anal Biochem 400(2): 207-212 (2010)): S-cirinarnoy Indole 13-enty; hexarelin (Demers A et al, Biochem J. 382(Pt 2):417-24(2004)); synthetically engineered nanoblockers (Chnari E, et al, Biomacromolecules. 7(6): 1796-805(2006)); as well as other small molecules, antibodies, and inhibitory nucleic acids, and methods for administering the same are known in the art; see, e.g., US20130116308, WO2014033130, and WO2012149465, inter alia.

FABP Inhibitors

FABP inhibitors and methods for administering the same are known in the art; see, e.g., U.S. Pat. No. 8,748,470. The FABP inhibitor can be a FABP4 and/or a FABP5 inhibitor. In some embodiments, the FABP inhibitor is a FABP4 inhibitor. The FABP4 inhibitor can be, e.g., a carbazole butanoic acid, aryl sulfonamide, sulfonylthiophene derivative, 4-hydroxypyrimidine, tetrahydrocarbazole derivative, 2,3-dimethylindole derivative, benzoylbenzene, biphenyl-alkanoic acid derivative, 2-oxazole-alkanoic acid derivative, tetrahydropyrimidone, pyridone, pyrazinone, aryl carboxylic acid, tetrazole, triazolopyrimidinone, and indole derivative. In certain aspects the FABP4 inhibitor is (BMS309403, Bristol Myers Squibb, described in Sulsky et al., Bioorganic & Medicinal Chemistry Letters 17(12), 3511-3515 (2007)); pyrazole, 4-{[2-(methoxycarbonyl)-5-(2-thienyl)-3-thienyl]amino}-4-oxo-2-butenoic acid or ((2′-(5-ethyl-3,4-diphenyl-1H-pyrazol-1-yl)(1,1′-biphenyl)-3-yl)oxy)-acetic acid. In some embodiments, the FABP inhibitor is a FABP5 inhibitor. The FABP5 inhibitor can be, e.g., an indole derivative (Lehmann et al 2004), triazolopyrimidinone derivative (Schering Corporation, PCT/US2009/063787), or Pyrazole. Other FABP5 inhibitors include SBFI26 (alpha-2,4-diphenylcyclobutane-1,3-dicarboxylic acid mono-1-naphthyl ester) and other α-truxillic acid derivatives, e.g., SBFI50 (alpha-2,4-diphenylcyclobutane-1,3-dicarboxylic acid mono-2-naphthyl ester), SBFI60 (alpha-2,4-diphenylcyclobutane-1,3-dicarboxylic acid mono-1-naphthyl amide), and SBFI62 (2,4-diphenylcyclobutane-1,3-dicarboxylic acid di-1-naphthyl amide), (Berger et al., PLoS One. 2012, 7(12), e50968; Kaczocha et al., PLoS One. 2014, 9(4), e94200.

In certain embodiments, the FABP4 inhibitor can be an inhibitory nucleic acid, e.g., as described herein, e.g., a small interference RNA (siRNA), in particular, a small hairpin RNA (shRNA). In certain aspects, the shRNA against FABP4 comprises a nucleic acid sequence of (5'-AUACUGAGAUUUCCUUCAU-3′) SEQ ID NO: 1 (Harj es et al., Oncogene. 2017 Feb. 16; 36(7): 912-921).

CPT1 Inhibitors

CPT1 inhibitors and methods for administering the same are known in the art; see, e.g., US2016/0102058.

CPT1 inhibitors include 2-(6-(4-chlorophenoxy)-hexyl)-oxirane-2-carboxylic acid ethyl ester (etomoxir) and its analog 2-tetradecylglycidic acid (TDGA) (Morillas et al., Biochem. J. (2000) 351, 495-502); aminocarnitine and ST1326 (Teglicar), a long-chain carbamoyl aminocarnitine derivative (Giannessi et al., J Med Chem 2003; 46: 303-309); and perhexiline (2-(2,2-dicyclohexylethyl)piperidine) (Horgan et al., U.S. Pat. No. 4,191,828; Horgan et al., U.S. Pat. No. 4,069,222). A number of derivatives of perhexiline are known in the art, including N-Substituted derivatives (see, e.g., WO 2007/096251); derivatives in which the piperidine has been replaced by other amine-bearing groups (see, e.g., LeClerc et al., J. Med. Chem., Vol. 25, pp. 709-714, 1982); derivatives in which the carbon separating the two cyclohexyl groups has been substituted with a hydroxyl group (see, e.g., Tilford and van Campen, J. Am. Chem. Soc., Vol. 76, pp. 2431-2441, 1954); deuterated derivatives (see, e.g., Schou, J. Label. Compd Radiopharm., Vol. 53, pp. 31-35, 2010); 4-monohydroxy metabolites (see, e.g., Davies et al., J. Chromatog. B, Vol. 843, pp. 302-309, 2006); and fluoro-perhexiline (FPER) compounds (US2016/0102058). A number are commercially available, including hydroxyperhexiline (i.e., 4-[1-(cyclohexyl)-2-(2-piperidinyl)ethyl]cyclohexanol; CAS Registry No 89787-89-3); trans-hydroxyperhexiline (i.e., trans-4-[1-(cyclohexyl)-2-(2-piperidinyl)ethyl]cyclohexanol; CAS Registry No 917877-74-8); and cis-hydroxyperhexiline (i.e., cis-4-[1-(cyclohexyl)-2-(2-piperidinyl)ethyl]cyclohexanol; CAS Registry No 917877-73-7). Additional examples of carnitine-palmitoyl-transferase-1 (CPT-1) inhibitors include 2-(6-(4-chlorophenoxy)hexyl)-oxirane-2-carboxylic acid ethyl ester (etomoxir), 2-(6-(4-difluoromethoxyphenoxy)hexyl)-oxirane-2-carboxylic acid ethyl ester, 2-(5-(4-difluoromethoxyphenoxy)pentyl)-oxirane-2-carboxylic acid ethyl ester, and 2-(5-(4-acetylphenoxy)pentyl)-oxirane-2-carboxylic acid ethyl ester, sodium-2(5-(4-chlorophenyl)pentyl-oxirane-2-caboxylate (Clomoxir), perhexiline, trimetazidine, sodium-4-hydroxyphenylglycine (Oxfenicine), 2-tetradecylglycidate (TDGA) (WO2003/037323A2).

Inhibitors of Mitochondrial Fatty Acid Beta-Oxidation

Examples of inhibitors of mitochondrial beta-oxidation of fatty acids include methyl palmoxirate, metoprolol, hydrazonopropionic acid, 4-bromocrotonic acid, ranolazine, hypoglycin, dichloroacetate, methylene cyclopropyl acetic acid, or beta-hydroxybutyrate (U.S. Pat. No. 7,510,710). Other examples of inhibitors of fatty acid beta-oxidation include pirprofen (Geneve et al., J Pharmacol Exp Ther. 1987; 242:1133-1137) and amiodarone (Fromenty et al., J Pharmacol Exp Ther.” 1990; 255:1371-6).

Inhibitory Nucleic Acids

Inhibitory nucleic acids useful in the present methods and compositions include antisense oligonucleotides, ribozymes, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics that hybridize to at least a portion of the target nucleic acid and modulate its function. In some embodiments, the inhibitory nucleic acids include antisense RNA, antisense DNA, chimeric antisense oligonucleotides, anti sense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., WO 2010040112.

Exemplary sequences for the target nucleic acids include the following.

| Gene | Nucleic Acid |
|---|---|
| CD36 (platelet | NM_000072.3, NM_001001547.2, |
| glycoprotein 4) | NM_001001548.2, NM_001127443.1, |
| | NM_001127444.1, NM_001289908.1, |
| | NM_001289909.1, NM_001289911.1 |
| FABP4 | NM_001442.2 |
| FABP5 | NM_001444.3 |
| CPT1 (CPT1A) | NM_001031847.2; NM_001876.4 |

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a target sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this disclosure, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

Antisense

In some embodiments, the inhibitory nucleic acids are antisense oligonucleotides. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to an RNA. Thus, oligonucleotides are chosen that are sufficiently complementary to the target, i.e., that hybridize sufficiently well and with sufficient specificity, to give the desired effect.

siRNA/shRNA

In some embodiments, the nucleic acid sequence that is complementary to a target RNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and anti sense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc NatlAcadSci USA 99:6047-6052, (2002).

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

Ribozymes

Trans-cleaving enzymatic nucleic acid molecules can also be used; they have shown promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional.

In general, enzymatic nucleic acids with RNA cleaving activity act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R. Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al, 1994, TIBTECH 12, 268; Bartel et al, 1993, Science 261:1411-1418; Szostak, 1993, TIM 17, 89-93; Kumar et al, 1995, FASEB J., 9, 1183; Breaker, 1996, Curr. Op. Biotech., 1, 442). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 rnM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

Modified Inhibitory Nucleic Acids

In some embodiments, the inhibitory nucleic acids used in the methods described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012. 22: 344-359; Nowotny et al., Cell, 121:1005-1016, 2005; Kurreck, European Journal of Biochemistry 270:1628-1644, 2003; FLuiter et al., Mol Biosyst. 5(8):838-43, 2009). In some embodiments, the oligonucleotide is a mixmer (includes alternating short stretches of LNA and DNA; Naguibneva et al., Biomed Pharmacother. 2006 November; 60(9):633-8; Ørom et al., Gene. 2006 May 10; 3720:137-41). Representative United States patents that teach the preparation of such hybrid structures comprise, but are not limited to, U.S. Pat. Nos. 5,013,830; 5,149,797; 5, 220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH2-NH—O—CH2, CH, ˜N(CH3)˜O˜CH2 (known as a methylene(methylimino) or MMI backbone], CH2-O—N (CH3)-CH2, CH2-N(CH3)-N(CH3)-CH2 and O—N(CH3)-CH2-CH2 backbones, wherein the native phosphodiester backbone is represented as O—P—O—CH); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃ OCH₃, OCH₃ O(CH₂)n CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-0-CH₂CH₂OCH₃, also known as 2′-O-(2-methoxyethyl)] (Martin et al, Helv. Chim. Acta, 1995, 78, 486). Other preferred modifications include 2′-methoxy (2′-0-CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-′7′7; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications’, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the inhibitory nucleic acids are chemically linked to one or more moieties or conjugates that enhance the activity, cellular distribution, or cellular uptake of the oligonucleotide. Such moieties comprise but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Locked Nucleic Acids (LNAs)

In some embodiments, the modified inhibitory nucleic acids used in the methods described herein comprise locked nucleic acid (LNA) molecules, e.g., including [alpha]-L-LNAs. LNAs comprise ribonucleic acid analogues wherein the ribose ring is “locked” by a methylene bridge between the 2′-oxgygen and the 4′-carbon—i.e., oligonucleotides containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herein.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of the LNA; for example, a series of oligonucleotides of 10-30 nucleotides spanning the length of a target RNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of more than four LNA residues, should be avoided where possible (for example, it may not be possible with very short (e.g., about 9-10 nt) oligonucleotides). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly.

Recombinant nucleic acid sequences can be individually isolated or cloned and tested for a desired activity. Any recombinant expression system can be used, including e.g. in vitro, bacterial, fungal, mammalian, yeast, insect or plant cell expression systems.

Nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors capable of expressing the nucleic acids of the invention can be delivered as described herein, and persist in target cells (e.g., stable transformants).

Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-0 atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y. (1993).

Pharmaceutical Compositions

The methods described herein can include the administration of pharmaceutical compositions and formulations comprising inhibitors or inhibitory nucleic acid sequences as described herein.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions of the invention include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered topically or transdermally, by a topical route, formulated as applicator sticks, solutions, suspensions, emulsions, gels, creams, ointments, pastes, jellies, paints, powders, and aerosols.

In some embodiments, compositions for transdermal application can further comprise cosmetically-acceptable carriers or vehicles and any optional components. A number of such cosmetically acceptable carriers, vehicles and optional components are known in the art and include carriers and vehicles suitable for application to skin (e.g., sunscreens, creams, milks, lotions, masks, serums, etc.), see, e.g., U.S. Pat. Nos. 6,645,512 and 6,641,824. In particular, optional components that may be desirable include, but are not limited to absorbents, anti-acne actives, anti-caking agents, anti-cellulite agents, anti-foaming agents, anti-fungal actives, anti-inflammatory actives, anti-microbial actives, anti-oxidants, antiperspirant/deodorant actives, anti-skin atrophy actives, anti-viral agents, anti-wrinkle actives, artificial tanning agents and accelerators, astringents, barrier repair agents, binders, buffering agents, bulking agents, chelating agents, colorants, dyes, enzymes, essential oils, film formers, flavors, fragrances, humectants, hydrocolloids, light diffusers, nail enamels, opacifying agents, optical brighteners, optical modifiers, particulates, perfumes, pH adjusters, sequestering agents, skin conditioners/moisturizers, skin feel modifiers, skin protectants, skin sensates, skin treating agents, skin exfoliating agents, skin lightening agents, skin soothing and/or healing agents, skin thickeners, sunscreen actives, topical anesthetics, vitamin compounds, and combinations thereof.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the invention can be administered for prophylactic and/or therapeutic treatments. In some embodiments, for therapeutic applications, compositions are administered to a subject who is need of reduced triglyceride levels, or who is at risk of or has a disorder described herein, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to decrease levels of T_{RM} in the tissue.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of therapeutic effect generated after each administration (e.g., effect on tumor size or growth), and the like. The formulations should provide a sufficient quantity of active agent to effectively treat, prevent or ameliorate conditions, diseases or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 21st ed., 2005.

Various studies have reported successful mammalian dosing using complementary nucleic acid sequences. For example, Esau C., et al., (2006) Cell Metabolism, 3(2):87-98 reported dosing of normal mice with intraperitoneal doses of miR-122 antisense oligonucleotide ranging from 12.5 to 75 mg/kg twice weekly for 4 weeks. The mice appeared healthy and normal at the end of treatment, with no loss of body weight or reduced food intake. Plasma transaminase levels were in the normal range (AST 3/4 45, ALT 3/4 35) for all doses with the exception of the 75 mg/kg dose of miR-122 ASO, which showed a very mild increase in ALT and AST levels. They concluded that 50 mg/kg was an effective, non-toxic dose. Another study by Krutzfeldt J., et al., (2005) Nature 438, 685-689, injected anatgomirs to silence miR-122 in mice using a total dose of 80, 160 or 240 mg per kg body weight. The highest dose resulted in a complete loss of miR-122 signal. In yet another study, locked nucleic acids (“LNAs”) were successfully applied in primates to silence miR-122. Elmen J., et al., (2008) Nature 452, 896-899, report that efficient silencing of miR-122 was achieved in primates by three doses of 10 mg kg-1 LNA-antimiR, leading to a long-lasting and reversible decrease in total plasma cholesterol without any evidence for LNA-associated toxicities or histopathological changes in the study animals.

In some embodiments, the methods described herein can include co-administration with other drugs or pharmaceuticals, e.g., compositions for providing cholesterol homeostasis. For example, the inhibitory nucleic acids can be co-administered with drugs for treating or reducing risk of a disorder described herein.

Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present.

Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### EXAMPLES

The following Methods were used in the examples set forth below.

Mice

Wide-type (WT) C57BL/6, CD45.1⁺, Thy1.1⁺, Rag1^{-/-} and μMT mice were purchased from Jackson Laboratory. Thy1.1⁺ Rag1⁻⁻ OT-I mice were maintained through routine breeding in the animal facility of Harvard Institute of Medicine, Harvard Medical School. Fabp4^{-/-}, Fabp5^{-/-} and Fabp4/5 dKO mice were kindly provided by Gokhan S. Hotamisligil (Harvard T. H. Chan School of Public Health). Mice were bred to generate Thy1.1⁺ CD45.1⁺ WT; Thy1.1⁺ CD45.2⁺ Fabp4/5 dKO; Thy1.1⁺ CD45.1⁺ WT OT-I, Thy1.1⁺ CD45.2⁺ Fabp4^{-/-} Fabp5^{+/-} OT-I, Thy1.1⁺ CD45.2⁺ Fabp4^{-/-} OT-I, Thy1.1⁺ CD45.2⁺ Fabp5^{-/-} OT-I and Thy1.1⁺ CD45.2⁺ Fabp4/5 dKO OT-I mice. Animal experiments were performed in accordance with the guidelines put forth by the Center for Animal Resources and Comparative Medicine at Harvard Medical School, and all protocols and experimental plans were approved by the HMS IACUC in advance of their performance. Mice were randomly assigned to each group before start and experiments were performed blinded with respect to treatment. For survival experiments, mice that had lost over 25% of original BW were euthanized.

Viruses and Infections

Recombinant VACV expressing OT-I T cell epitope Ova₂₅₇₋₂₆₄ and Western Reserve strain (WR-VACV) were originally obtained from B. Moss (NIH). Virus was expanded and titered by standard procedures as described previously⁹. 2×10⁶ p.f.u. of VACV_{OVA} was used for infection by either skin scarification or intra-tracheal infection. 2×10⁶ p.f.u. WR-VACV was used at a lethal dose in intranasal infections, as described previously⁹.

Antibodies and Flow Cytometry

The following anti-mouse antibodies were obtained from BD PharMingen: PerCP-conjugated anti-CD3e (553067), PE-conjugated anti-CD8 (557654), PE-Cy7-conjugated anti-CD8 (552877), APC-Cy7-conjugated anti-CD8 (557654), PE-conjugated anti-Thy1.1 (561404), APC-conjugated anti-Thy1.1 (557266), Alexa Fluor 488-conjugated anti-KLRG1 (561619), PE-conjugated anti-integrin α4β7 (553811), PE-Cy7-conjugated anti-CD62L (560516), APC-Cy7-conjugated CD62L (560514), APC-conjugated anti-IFN-γ (554413). Biolegend: Alexa Fluor 488-conjugated anti-CD3e (100321), Alexa Fluor 647-conjugated anti-CD4 (100424), Alexa Fluor 594-conjugated anti-Ep-CAM (118222), FITC-conjugated anti-CD45.1 (110706), PE-conjugated anti-CD45.1 (110708), PE-Cy7-conjugated anti-CD45.2 (109830), APC-conjugated anti-CD45.2 (109814), PE-conjugated anti-CD45.2 (109808), APC-conjugated anti-KLRG1 (138412), PE-conjugated anti-CD44 (103008), PE-Cy7-conjugated anti-CD44 (103030), PE-Cy7-conjugated anti-CD69 (104512), APC-conjugated anti-CD103 (121414), APC-conjugated anti-integrin α4β7 (120608). The following anti-human antibodies were obtained from Biolegend: APC-Cy7-conjugated anti-CD3 (300425), PerCp-conjugated anti-CD4 (317431), APC-conjugated anti-CD8 (300911), Alexa Fluor 488-conjugated anti-CD69 (310916), PE-Cy7-conjugated anti-CD69 (310911), PE-conjugated anti-CD62L (304805), FITC-conjugated anti-CD45RO (304204). Abcam: anti-human FABP4 (9B8D). NOVUS: Alexa Fluor 405-conjugated anti-human FABP5 (FAB3077V). PE-conjugated B8R20-27/H-2 Kb pentamers were obtained from ProImmune Ltd, and stained according to the manufacturer's protocol. E- or P-selectin ligand expression was examined by incubating cells with rmE-Selectin/Fc Chimera (575-ES; R&D System) or rmP-Selectin/Fc Chimera (737-PS; R&D System) in conjunction with PerCP-conjugated F(ab′)2 fragments of goat anti-human IgG F(c) antibody (109-126-170; Jackson ImmunoResearch). To measure uptake of Bodipy conjugated palmitate (Bodipy FL C16; D-3821; Thermo Fisher) ex vivo, cells were incubated for 30 minutes at 37° C. with 1 μM Bodipy FL C16 in PBS with 20 μM FA-free BSA (A8806; Sigma-Aldrich). Bodipy uptake was quenched by adding 4× volume of ice-cold PBS with 2% FBS and then cells washed twice prior to flow cytometry analysis. Annexin V staining was included to exclude dead/dying cells during FACS data acquisition. To block Bodipy uptake, cells were incubated with 100 μM palmitic acid (P0500; Sigma-Aldrich) for 10 minutes at 37° C. prior to Bodipy addition. Apoptosis was measured with FITC Annexin V Apoptosis Detection Kit (640922; Biolegend) according to manufacturer's protocol. Flow cytometry data were acquired with a FACS Canto II flow cytometer (BD Biosciences) and data were analyzed with Flowjo software (Tree Star).

Preparation of Cell Suspensions

Lymph nodes and spleen were harvested and pressed through a 70-μm nylon cell strainer to prepare cell suspensions. Red blood cells (RBC) were lysed using RBC lysis buffer (00-4333-57; eBioscience). Skin tissue was excised after hair removal, separated into dorsal and ventral halves, minced, and then incubated in Hanks balanced salt solution (HBSS) supplemented with 1 mg/ml collagenase A (11088785103; Roche) and 40 μg/ml DNase I (10104159001; Roche) at 37° C. for 30 min. After filtration through a 70-μm nylon cell strainer, cells were collected and washed three times with cold PBS before staining.

Mouse Adoptive Transfer and Treatment

Lymph nodes were collected from naive female donor mice at age of 6-8 weeks. T cells were purified by magnetic cell sorting using a mouse CD8α⁺ T-cell isolation kit (130-104-075; Miltenyi Biotec) or a mouse CD4⁺ T-cell isolation kit (130-104-454; Miltenyi Biotec), according to the manufacturer's protocols. T cells were then transferred intravenously into female recipient mice at a total number of 5×10⁵ or 2.5×10⁵ cells/population in co-transfer experiments where cell types were transferred at a ratio of 1:1. To generate mixed bone marrow chimeras, T cells- and NK cell-depleted Thy1.1⁺ CD45.1⁺ WT and Thy1.1⁺ CD45.2⁺ Fabp4/5 dKO bone marrow was mixed in a 1:1 ratio and transferred at a number of 1×10⁶/population into sublethally irradiated recipient mice. Mice were rested for eight weeks before infection for full reconstitution of T cells and restoration of an intact immune system. Rag1^{-/-} T-cell reconstituted mice were generated by adoptive transfer of 3.5×10⁶ CD4⁺ with 2×10⁶ CD8⁺ WT or CD8⁺ Fabp4/5 dKO cells. T cells were labeled with carboxyfluorescein succinimidyl ester (CFSE, 65-0850; eBioscience) before co-transfer, where indicated. In some experiments, mice were treated daily with FTY720 (10006292; CAYMAN, 1 mg/kg) by intraperitoneal injection or with etomoxir (E1905; Sigma-Aldrich, 1 μg/site), GW9662 (M6191, Sigma-Aldrich, 1 mg/kg) or trimetazidine (653322, Sigma-Aldrich, 1 mg/kg) by intradermal injection.

Microarray, Data Analysis and Quantitative Real-Time PCR

For each group of microarray dataset, OT-I cells from 15-20 mice were sorted with a FACSAria III (BD Biosciences) and pooled. RNA was extracted with an RNeasy Micro kit (74004; Qiagen). RNA quality and quantity was assessed with a Bioanalyzer 2100 (Agilent). Then RNA was amplified and converted into cDNA by a linear amplification method with WT-Ovation Pico System (3302-60; Nugen). Subsequently cDNA was labeled with the Encore Biotin module (4200-60; Nugen) and hybridized to GeneChip MouseGene 2.0 ST chips (Affymetrix) at the Translational Genomics Core of Partners Healthcare, Harvard Medical School. GeneChips were scanned using the Affymetrix GeneChip Scanner 3000 7G running Affymetrix Gene Command Console ver 3.2. The data were analyzed by using Affymetrix Expression Console ver 1.3.0.187 using Analysis algorithm RMA. To evaluate overall performance of microarray data, principal component analysis (PCA) and Pearson correlation coefficients among 12 diverse samples were applied by using 26,662 transcripts (R Program). All microarray data was submitted to the Gene Expression Omnibus (accession code GSE 79805).

For relative quantitative real-time PCR, RNA was prepared as described above. Bio-Rad iCycler iQ Real-Time PCR Detection System (Bio-Rad) was used with the following settings: 45 cycles of 15 s of denaturation at 95° C., and 1 min of primer annealing and elongation at 60° C. Real-time PCR was performed with 1 μl cDNA plus 12.5 μl of 2×iQ SYBR Green Supermix (Bio-Rad) and 0.5 μl (10 μM) specific primers: mouse Fabp4 forward (5′-TTT CCT TCA AAC TGG GCG TG-3′) and mouse Fabp4 reverse (5′-CAT TCC ACC ACC AGC TTG TC-3′); mouse Fabp5 forward (5′-AAC CGA GAG CAC AGT GAA G-3′) and mouse Fabp5 reverse (5′-ACA CTC CAC GAT CAT CTT CC-3′); mouse Pparγ forward (5′-TCG CTG ATG CAC TGC CTA TG-3′) and mouse Pparγ reverse (5′-GAG AGG TCC ACA GAG CTG ATT-3′); mouse β-actin forward (5′-CAT TGC TGA CAG GAT GCA GAA GG-3′) and mouse β-actin reverse (5′-TGC TGG AAG GTG GAC AGT GAG G-3′). For absolute quantitative real-time PCR. each standard curve was constructed using 10-fold serial dilutions of target gene template ranging from 10⁷ to 10² copies per mL and obtained by plotting values of the logarithm of their initial template copy numbers versus the mean Ct values. The actual copy numbers of target genes were determine by relating the Ct value to a standard curve.

Immunofluorescence Microscopy

Mice were perfused with buffer A (0.2 M NaH₂PO₄, 0.2M Na₂HPO₄, 0.2 M L-lysine and 0.1 M sodium periodate with 2% paraformaldehyde) and infected skin sites were harvested and incubated for 30 min on ice in buffer A. Skin tissue was washed twice with PBS and incubated for 30 min at 4° C. in 20% sucrose. Fixed tissue was embedded in OCT (Tissue Tek IA018; Sakura) and frozen in liquid nitrogen. Skin sections were performed on a cryostat (Leica CM1850 UV) at 6-μm thickness and air-dried for 6-8 h. Sections were then fixed in −20° C. acetone for 5 min, rehydrated with PBS, and blocked with 2% FCS in PBS for 15 min at room temperature (20° C.). Sections were stained with rabbit anti-mouse/human FABP4 antibody (EPR3579; ab92501, Abcam), rabbit anti-mouse/human FABP5 (H-45, sc-50379, Santa Cruz) overnight at 4° C. in a semi-humid chamber. Sections were rinsed for 10 min in PBS, and labeled with donkey anti-rabbit Rhodamine Red™-X (711-296-152; Jackson ImmunoResearch) for 1 hr at room temperature (20° C.). Sections were rinsed for 10 min in PBS, and stained with Alexa Fluor647-conjugated anti-mouse Thy1.1 (202508, Biolegend) in PBS for 1 hr at room temperature. Then sections were rinsed three times (for 5 min each time) with TBS-Tween 20 by shaking and mounted with ProLong Diamond Antifade Mountant with DAPI (P36962; ThermoFisher). For tissue lipid visualization, sections were stained with BODIPY® 493/503 (4,4-Difluoro-1,3,5,7,8-Pentamethyl-4-Bora-3a,4a-Diaza-s-Indacene) (D3922, Molecular Probes) before mounted. Images were acquired with Leica TCS SP8 confocal microscopy (Harvard NeuroDiscovery Center Optical Imaging Core) and analyzed with Image.

Lentiviral siRNA Transduction

Scrambled, Pparγ and Cpt1a siRNA GFP lentiviruses were purchased from ABM (Applied Biological Materials Inc., Canada) with sequences as following: scrambled siRNA: GGG TGA ACT CAC GTC AGA A; Pparγ KD1: AAT ATG ACC TGA AGC TCC AAG AAT A; Pparγ KD2: GTC TGC TGA TCT GCG AGC C; Cpt1a KD1: GGA GCG ACT CTT CAA TAC TTC CCG CAT CC, Cpt1a KD2: GGT CAT AGA GAC ATC CCT AAG CAG TGC CA.

For siRNA lentivirus transduction, OT-I mice were infected with 2×10⁶ VACV_{OVA} by skin scarification. At 60 hr later, CD8⁺ T cells were harvested from draining lymph nodes and incubated in medium with 10 μg/ml polybrene and 20 ng/ml hIL-2 at 37° C. for 30 min. Then cells were infected with scrambled, Pparγ or Cpt1a siRNA GFP lentiviruses, respectively, in presence of ViralPlus Transduction Enhancer G698 at 1:100 in order to enhance transduction efficiency. For adoptive transfer, 2.5×10⁵ Pparγ or Cpt1a siRNA transduced OT-I cells (together with the same number of congenically scrambled siRNA transduced OT-I cells) were co-transferred into recipient mice that were previously infected with 2×10⁶ VACV_{OVA} by skin scarification. At 40 days later, mice were sacrificed and the number of siRNA transduced OT-I cells in the infected skin tissue were isolated and enumerated by flow cytometry based on the GFP marker. Recipient and both donor populations used for co-transfers differed in CD90 and CD45 alleles (being CD90.2/45.2, CD90.1/45.1 or CD90.1/45.2—the combinations differing between experiments) to allow for identification of donor populations.

FAO Assay

Oxidation of exogenous free fatty acids (FFA) was measured using XF Palmitate-BSA FAO substrate with XF cell mito stress kit according to the manufacturer's protocol (Seahorse Bioscience). Freshly isolated and sorted T cells (2.5×10⁵) were incubated for 30 min with FAO assay medium (111 mM NaCl, 4.7 mM KCl, 1.25 mM CaCl₂), 2.0 mM MgSO₄, 1.2 mM Na₂HPO₄, 2.5 mM glucose, 0.5 mM carnitine and 5 mM HEPES). When required, cells were pre-treated with etomoxir (40 μM) for 15 min. Afterwards, BSA (34 μM) or palmitate-BSA (200 μM palmitate conjugated with 34 μM BSA) was added to the medium, and the oxygen-consumption rate (OCR) was measured under basal conditions and in response to 1 uM oligomycin, 1.5 uM fluorocarbonyl cyanide phenylhydrazone (FCCP), and 100 nM rotenone+1 uM antimycin A. Results were normalized to those of control cells in the presence of BSA.

Determination of Viral Load

VACV load was evaluated by quantitative real-time PCR as described previously³. In brief, 6 days after re-infection, inoculated skin samples were harvested and DNA was purified with the DNeasy Mini Kit (51304; Qiagen) according to the manufacturer's protocol. Real-time PCR was performed with the Bio-Rad iCycler iQ Real-Time PCR Detection System (Bio-Rad Laboratories). The primers and TaqMan probe used in the quantitative PCR assay are specific for the ribonucleotide reductase Vv14L of VACV. The sequences are (forward) 5′-GAC ACT CTG GCA GCC GAA AT-3; (reverse) 5′-CTG GCG GCT AGA ATG GCA TA-3; (probe) 5′-AGC AGC CAC TTG TAC TAC ACA ACA TCC GGA-3′. The probe was 5′-labeled with FAM and 3′-labeled with TAMRA (Applied Biosystems, Foster City, Calif.). Amplification reactions were performed in a 96-well PCR plate (Bio-Rad Laboratory) in a 20 μl volume containing 2× TaqMan Master Mix (Applied Biosystems), 500 nM forward primer, 500 nM reverse primer, 150 nM probe, and the template DNA. Thermal cycling conditions were 50° C. for 2 min and 95° C. for 10 min for one cycle, followed by 45 cycles of amplification (94° C. for 15 s and 60° C. for 1 min). To calculate the viral load, a standard curve was established from DNA of a VACV stock with previously determined titer. Corresponding CT values obtained by the real-time PCR methods were plotted on the standard curve to estimate viral load in the skin samples.

Intracellular Cytokine Detection

Infected skin was harvested 6 days post VACV_{OVA} re-infection and single cell suspensions were prepared as described above. Then cells were incubated with 2 μg/ml SINFEKL peptide of ovalbumin (RP 10611; GenScript) in the presence of Brefeldin A (00-4506-51; eBioscience) for 7 hr. Fc receptors were blocked with CD16/CD32 monoclonal antibodies (14-0161-82; eBioscience). Then intracellular IFN-γ (554413; BD) as well as IFN-γ isotype control (554686; BD) staining was performed using Intracellular Cytokine Detection Kits (BD Bioscience) according to manufacturer's instruction before acquisition on a flow cytometer.

Human Tissue Samples

This is an experimental laboratory study performed on human tissue samples. All studies were performed in accordance with the Declaration of Helsinki. Blood from healthy individuals was obtained after leukapheresis, and normal skin was obtained from healthy individuals undergoing cosmetic surgery procedures. Lesional skin from patients with psoriasis was obtained from patients seen at the Brigham and Women's Hospital or at Rockefeller University. All tissues were collected with informed consent (where applicable) and with prior approval from the Partners or Rockefeller Institutional Review Boards. Skin tissue was extensively minced and then incubated for 2 hr at 37° C. in RPMI-1640 containing 0.2% collagenase type I (Invitrogen) and 30 Kunitz Units/ml DNAse I (Sigma Aldrich). Thereafter, cells were collected by filtering the collagenase-treated tissue through a 40 μm cell strainer (Fisher Scientific) followed by two washes with culture medium to remove any residual enzyme. Cells were stained with directly conjugated Abs and analyzed by flow cytometry. Gate strategy: T_{N}, CD45RA⁺ CD45RO⁻ CD3⁺ CD8⁺ CD62L⁺; T_{CM}, CD45RO⁺ CD3⁺ CD8⁺ CD62L⁺CCR7⁺; T_{EM}, CD45RO⁺ CD3⁺ CD8⁺ CD62L⁻ CCR7⁻; T_{RM}, CD45RO⁺ CD3⁺ CD8⁺ CD62L⁻ CD69⁺. For immunofluorescent staining, skin tissues were embedded in OCT and frozen in liquid nitrogen immediately after surgery.

Statistical Analysis

Comparisons for two groups were calculated using Student's t test (two tailed). Comparisons for more than two groups were calculated with one-way analysis of variance (ANOVA) followed by Bonferroni's multiple comparison tests. Two-way ANOVA with Holm-Bonferroni post hoc analysis was used to compare weight loss between groups and Log-rank (Mantel-Cox) test was used for survival curves. p<0.05 was considered statistically significant.

Extracellular Fatty Acid Uptake.

To measure uptake of Bodipy conjugated palmitate (Bodipy FL C16; D-3821; Thermo Fisher) ex vivo, cells were incubated for 30 minutes at 37° C. with 1 μM Bodipy FL C16 in PBS with 20 μM FA-free BSA (A8806; Sigma-Aldrich). Bodipy uptake was quenched by adding 4× volume of ice-cold PBS with 2% FBS and then cells washed twice prior to flow cytometry analysis. Annexin V staining was included to exclude dead/dying cells during FACS data acquisition. To block Bodipy uptake, cells were incubated with 100 μM palmitic acid (P0500; Sigma-Aldrich) for 10 minutes at 37° C. prior to Bodipy addition.

### Example 1. Survival of Tissue-Resident Memory T Cells Requires Exogenous Lipid Uptake and Metabolism

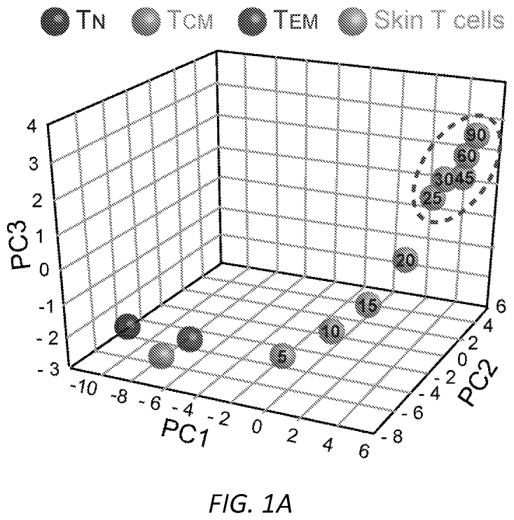

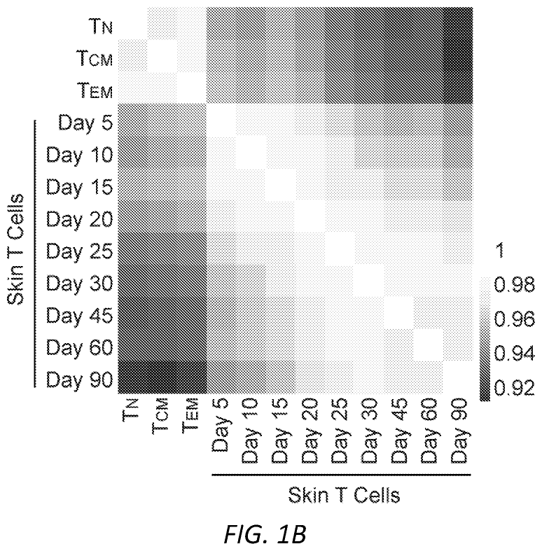

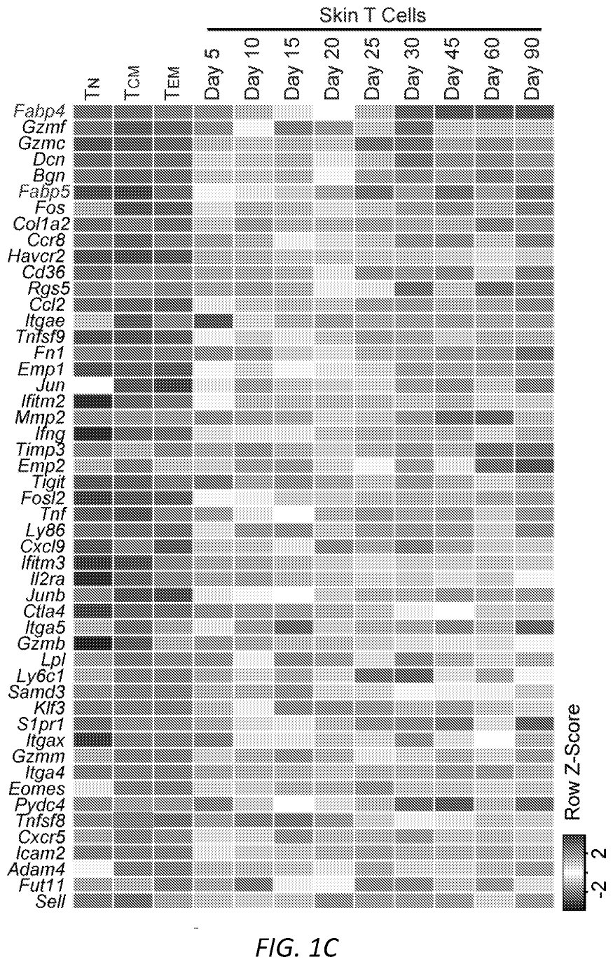

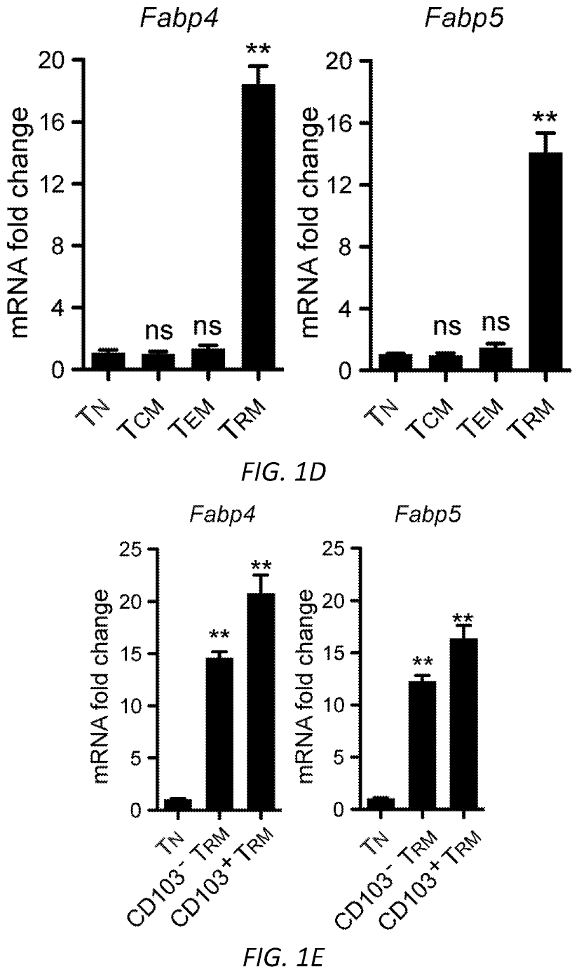

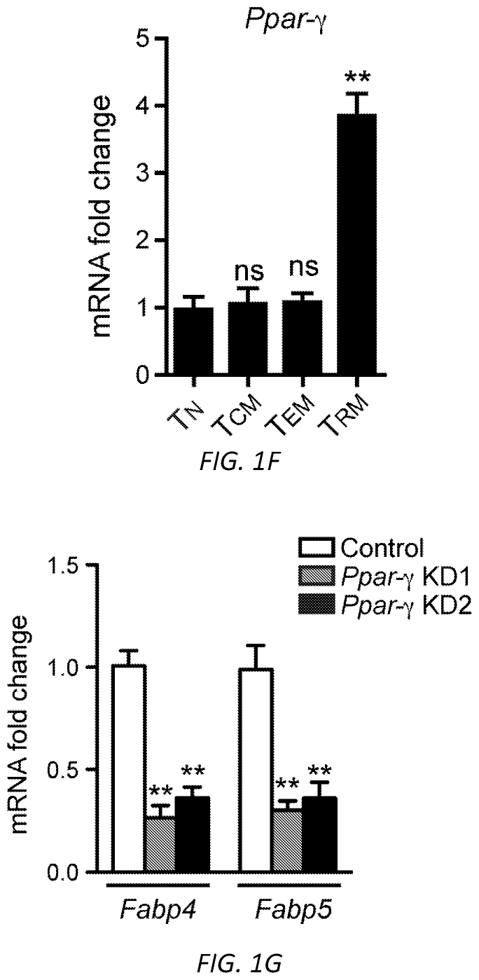

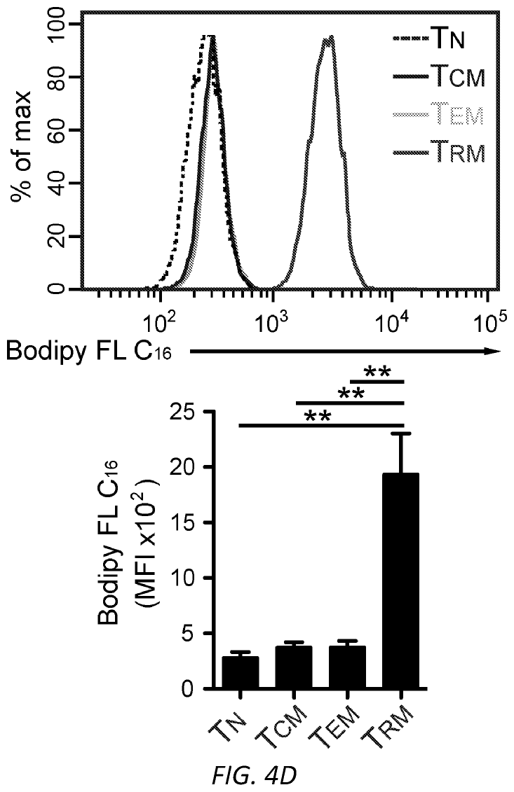

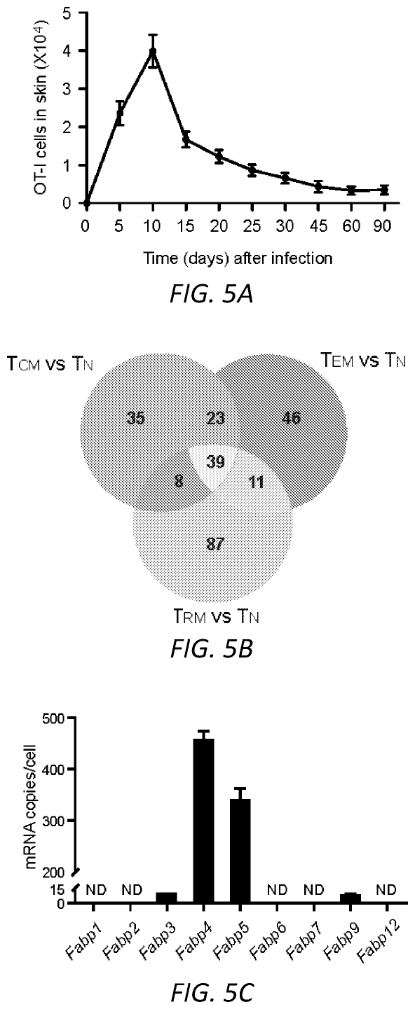

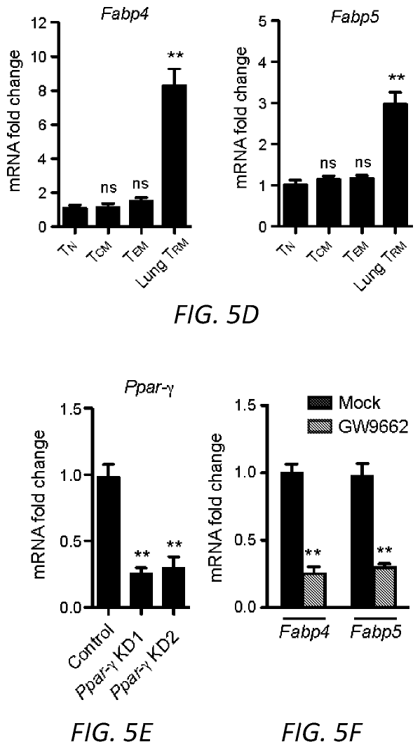

Although previous studies have yielded clues¹⁰⁻¹³, little is known about the molecular program that regulates the long-term survival of these cells. To answer this question, we first evaluated skin T_{RM} maturation by comparing the gene expression patterns at different timepoints after infection. OT-I transgenic T cells were transferred into recipient mice one day before immunization with a rVACV_{OVA}¹⁴. OT-I cells were readily found in the skin at day 5 post infection and reached their maximal level at day 10 before beginning to decrease in numbers (FIG. 5A). Skin-infiltrating OT-I cells were sorted at different timepoints after infection and were analyzed by transcriptional profiling. Principal-component analysis (PCA) showed that transcriptomes of skin infiltrating T cells clustered tightly from day 25 to day 90 post infection, suggesting that mouse skin CD8⁺ T_{RM} maturation is largely completed by day 25 post infection (FIG. 1A). Transcriptomes of T_{RM} are distinct from those of central memory T cells (T_{CM}) and effector memory T cells (T_{EM}), (FIG. 1A-B, FIG. 5B), consistent with previous reports¹¹⁻¹³.

Next, we directly compared T_{RM} (day 30) and T_{CM} (FIG. 1C). Strikingly, genes encoding fatty acid binding protein 4 (FABP4) and FABP5, were among the most strongly upregulated genes in T_{RM}, as was the gene for CD36, a lipid scavenger cell surface receptor¹⁵ (FIG. 1C). Quantitative real-time PCR confirmed this increased gene expression of Fabp4 and Fabp5 in CD8⁺ T_{RM} (FIG. 1D, FIG. 5C, FIG. 1E). Skin immunofluorescent staining (IF) showed expression of FABP4 and FABP5 protein in skin CD8⁺ T_{RM}. To extend these observations to other peripheral tissues, mice with transferred OT-I cells were infected with VACV_{OVA} by intra-tracheal infection and gene expression of Fabp4 and Fabp5 were measured 30 days later in lung CD8⁺ T_{RM}. Consistently, increased Fabp4 and Fabp5 gene expression was observed (FIG. 5D).

Peroxisome proliferator-activated receptors (PPAR) are adipogenic regulators that have been reported to influence Fabp4 and Fabp5 gene expression¹⁶. Pparγ, but not Pparα or Ppar®, was selectively up-regulated in T_{RM} compared to T_{N}, T_{CM} and T_{EM} (FIG. 1F). Knockdown of Pparγ expression using lentiviral siRNA, or treatment of mice with GW9662 (an irreversible PPARγ antagonist), inhibited Fabp4 and Fabp5 gene expression in CD8⁺ T_{RM} (FIG. 1G, FIG. 5E-F). These data indicate that PPARγ is an upstream regulator of Fabp4 and Fabp5 gene expression.

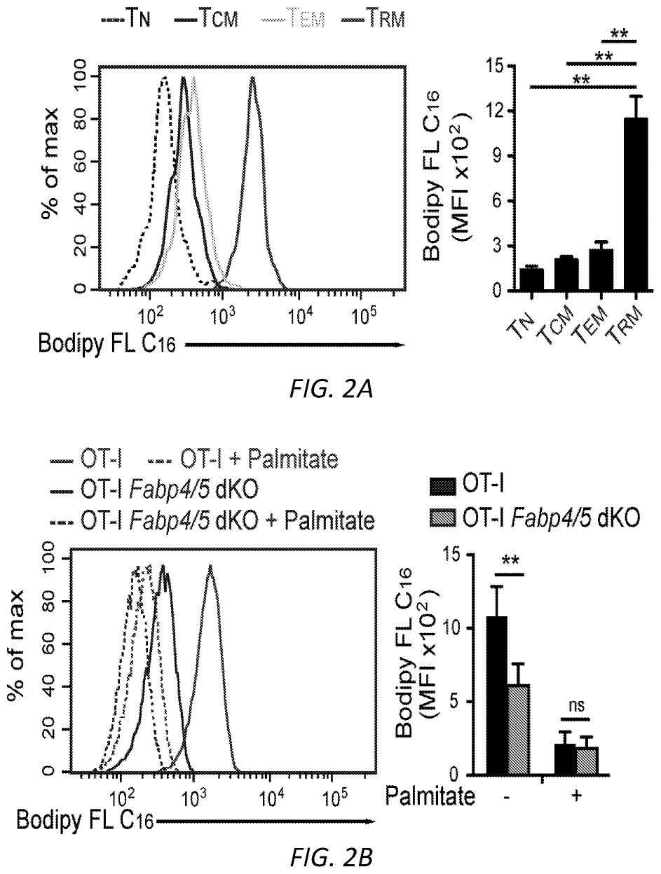

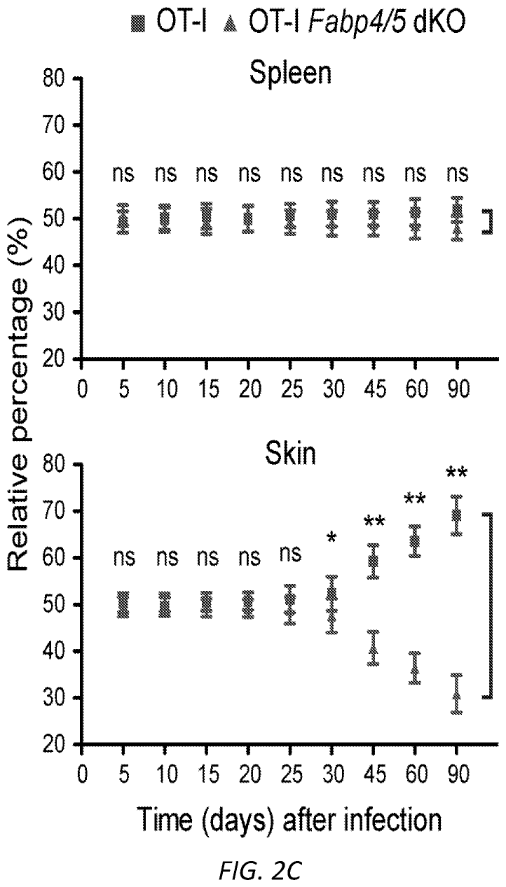

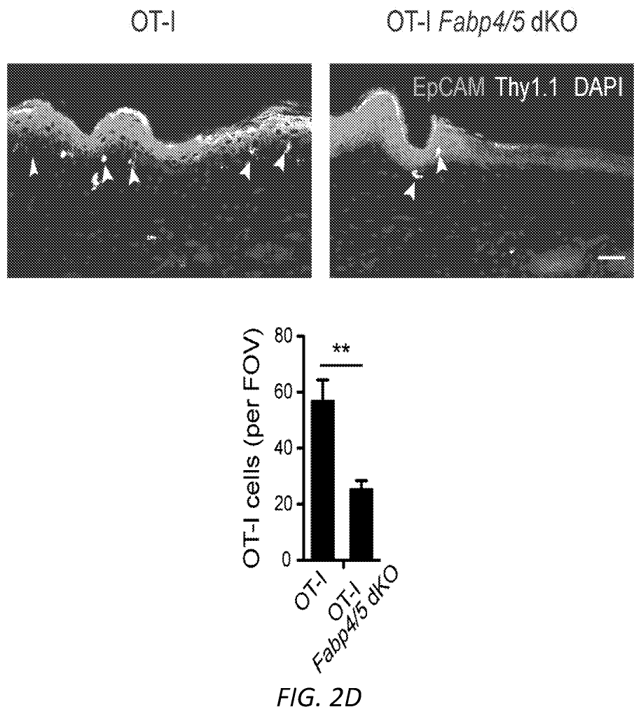

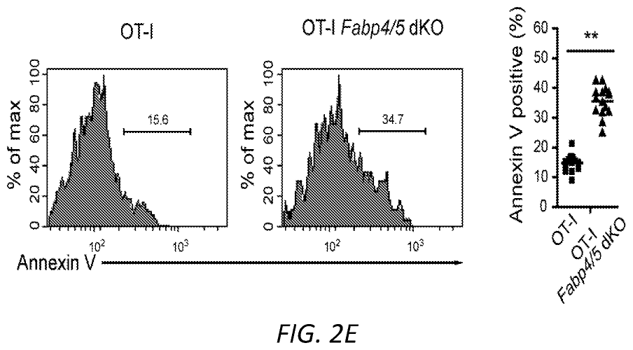

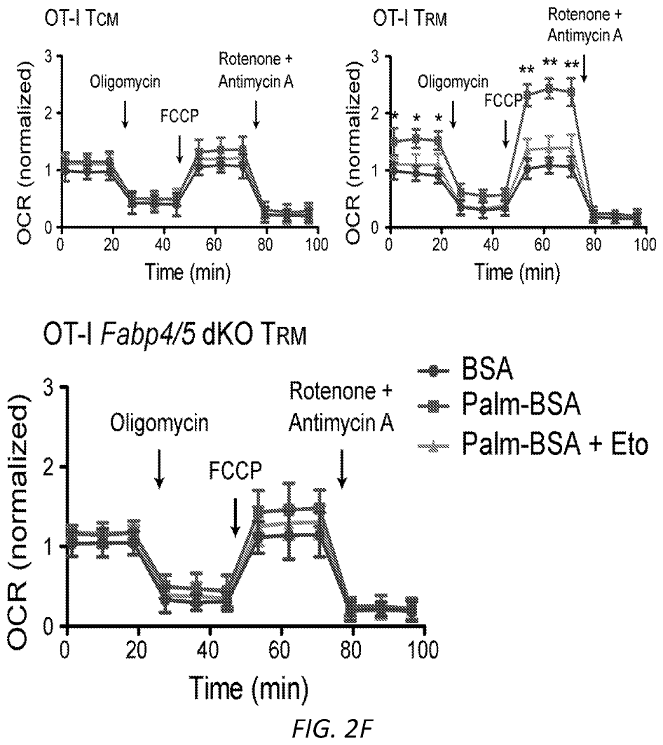

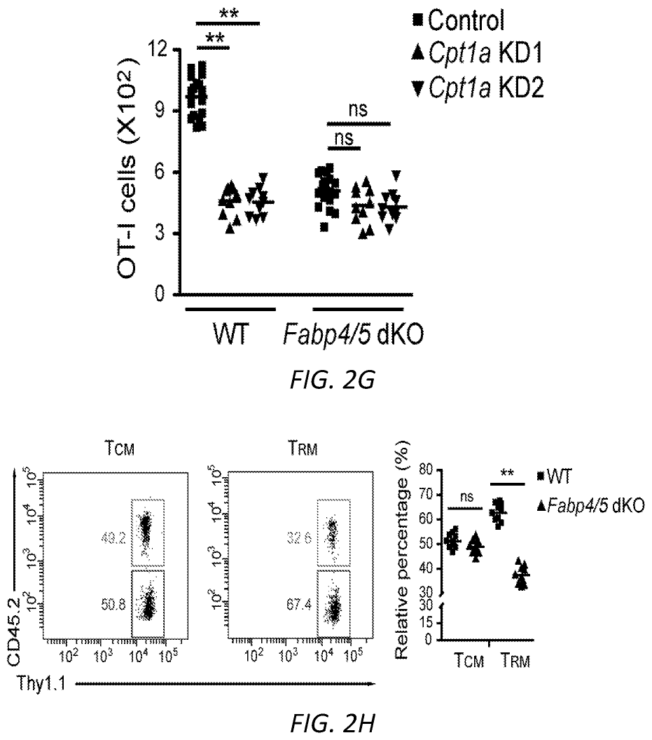

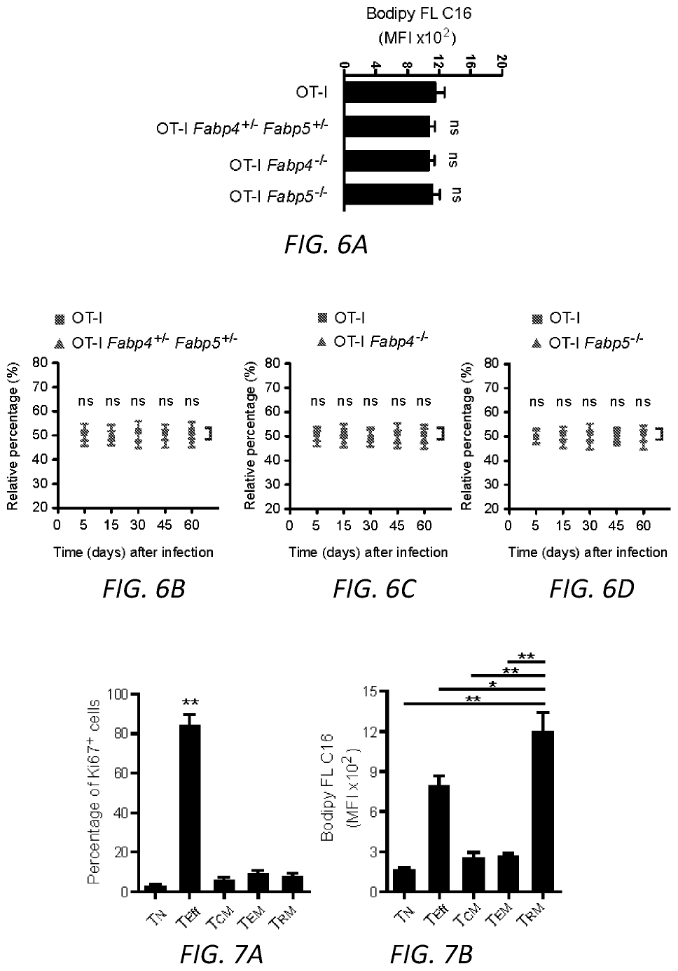

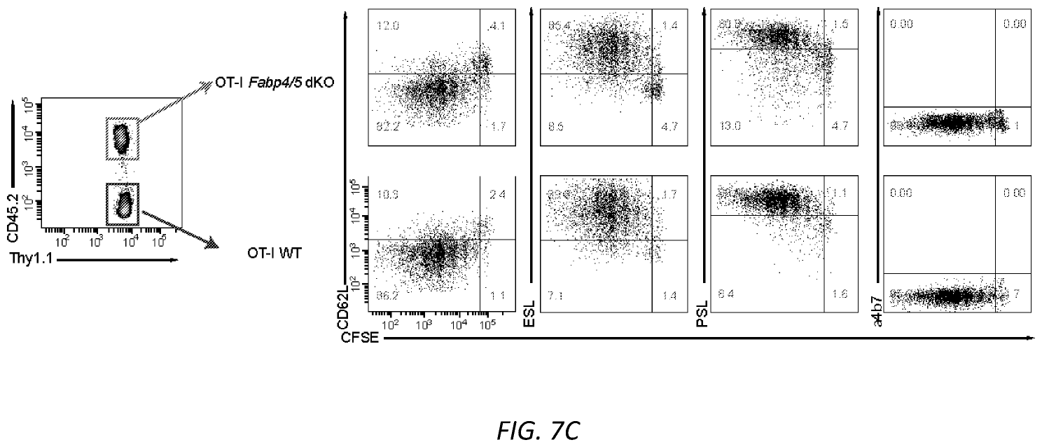

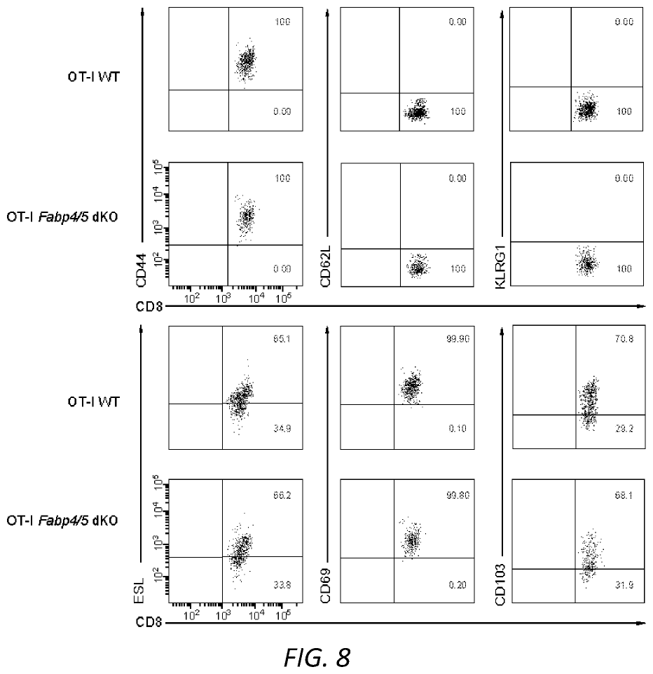

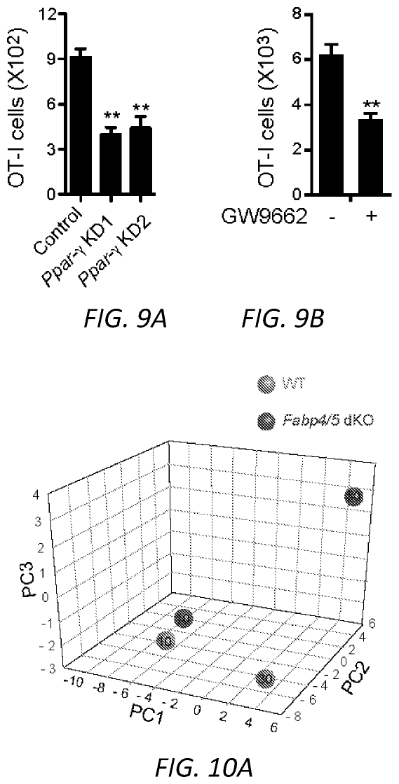

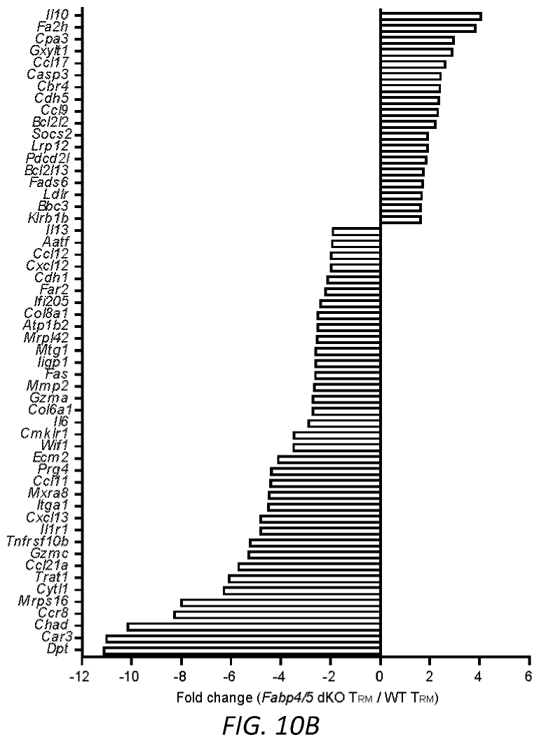

Upon activation, naive T cells undergo metabolic reprogramming as they proliferate and develop into different subsets of memory T cells^{17,18}. The strongly upregulated T_{RM} genes Fabp4 and Fabp5 encode for lipid chaperone proteins that bind to hydrophobic ligands to coordinate lipid uptake and intracellular trafficking¹⁹. Extracellular free fatty acid (FFA) could be visualized in mouse epidermis, where skin CD8⁺ T_{RM} localize³. Given the magnitude of their upregulation, we hypothesized that FABP4 and FABP5 might play a role in CD8⁺ T_{RM} physiology in skin. To test our hypothesis, we first compared the extracellular FFA uptake of OT-I memory T cell subtypes in vitro. Compared to T_{N}, T_{CM} and T_{EM} counterparts, substantially more Bodipy FL C16 was internalized by OT-I T_{RM} (FIG. 2A). FABP4 and FABP5 are highly homologous and bind to fatty acids with similar selectivity and affinity¹⁹. Given the compensatory and redundant role of these two molecules²⁰, we used mice lacking both Fabp4 and Fabp5 (Fabp4/5 dKO) to examine the contribution of these molecules to FFA uptake. By FACS analysis, OT-I Fabp4/5 dKO T_{RM} internalized much less fatty acid in vitro compared to WT counterparts (FIG. 2B), suggesting that FABP4 and FABP5 were important for the specific uptake of palmitate. We then asked whether loss of FABP4 and FABP5 would impair T_{RM} behavior in vivo. OT-I WT and OT-1 Fabp4/5 dKO cells were mixed at 1:1 ratio and transferred into congenic recipients. Mice were then infected with VACV_{OVA}, and the number of OT-I WT and OT-I Fabp4/5 dKO cells in different anatomic compartments was assessed. No difference was observed between the number of spleen WT OT-I and OT-I Fabp4/5 dKO T_{CM} at any timepoint, indicating that deficiency of FABP4 and FABP5 did not affect T_{CM} survival (FIG. 2C). However, OT-I Fabp4/5 dKO cells in skin displayed a profound defect in persistence, beginning 25 days post infection. The ratio of OT-I Fabp4/5 dKO/OT-1 WT T_{RM} cells declined steadily over time thereafter (FIG. 2C). Deficiency of both FABP4 and FABP5 also decreased numbers of OT-I T_{RM} detectable by IF without affecting their recruitment or tissue localization (FIG. 2D, FIG. 8). In contrast, OT-1 Fabp4^{-/-}, Fabp5^{-/-}, or Fabp4^{+/-} Fabp 5^{+/-} T_{RM} showed no defect in long-term survival (FIGS. 6A-D), consistent with the compensatory and redundant role of these two molecules²⁰. Highly proliferative OT-I effector T cells (T_{eff}) acquired more FFA compared to T_{N}, T_{CM} and T_{EM} but less FFA compared to T_{RM} (FIG. 7A-B). Interestingly, OT-I Fabp4/5 dKO T_{eff} displayed a similar proliferative capacity and tissue-homing receptor expression as WT T_{eff} at 60 hr post infection (FIG. 7C). These data indicate that absence of FABP4 and FABP5 does not affect CD8⁺ T cell proliferation or recruitment to skin. Inhibition of Fabp4/5 gene expression by knocking down Pparγ expression in OT-I cells, or inhibition of PPARγ by GW9662 treatment, decreased the long-term persistence of CD8⁺ T_{RM} in skin (FIGS. 9A-B). These data suggest a critical role of FABP4 and FABP5 for long-term survival CD8⁺ T_{RM} in skin. Annexin V staining comparing OT-I WT and Fabp4/5 dKO T_{RM} demonstrate that the latter had a higher ratio of apoptosis (FIG. 2E). The gene expression profile of Fabp4/5 dKO T_{RM} revealed that immune response genes were significantly down-regulated, while genes involved in anti-inflammatory responses and apoptosis were up-regulated compared to their WT counterparts (FIGS. 10A-B).

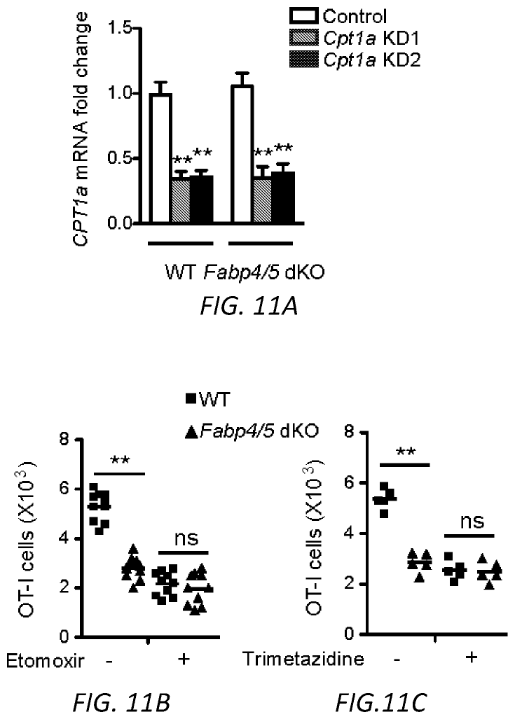

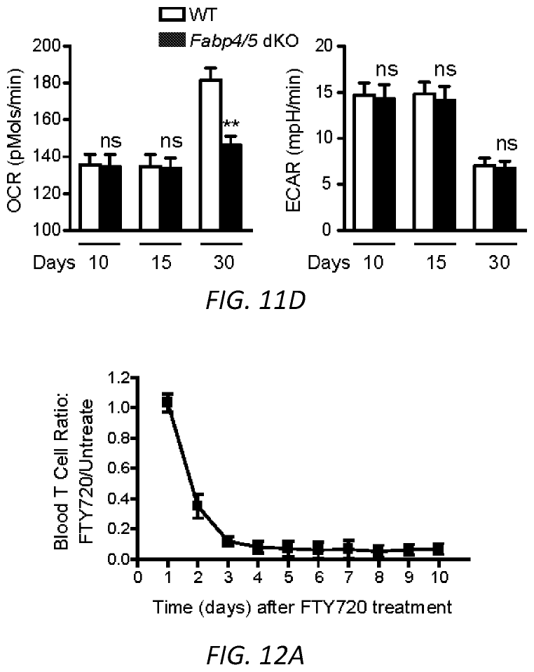

To determine the dependence of T_{RM} on exogenous FFA uptake for oxidative metabolism, we utilized the Seahorse Fatty Acid Oxidation (FAO) assay²¹. Addition of extracellular fatty acids induced a significantly higher basal and FCCP-stimulated maximal oxygen-consumption rate (OCR) in OT-I T_{RM} (FIG. 2F). This enhanced OCR was blocked by pre-treatment with etomoxir, an inhibitor of mitochondrial carnitine palmitoyltransferase 1a (CPT1A), an enzyme central to mitochondrial fatty acid β-oxidation²² (FIG. 2F). By contrast, T_{CM} or Fabp4/5 dKO T_{RM} OT-I cells did not demonstrate an increased OCR when supplied with exogenous fatty acid, and the addition of etomoxir had no effect on their cellular respiration (FIG. 2F). In vivo knockdown of Cpt1a or treatment of mice with either etomoxir or trimetazidine²³ decreased the number of OT-I WT T_{RM} to an extent similar to their Fabp4/5 dKO counterparts (FIG. 2G, FIGS. 11A-C).

These data suggest that skin CD8⁺ T_{RM} utilize oxidative metabolism of exogenous FFA to support their long-time survival. Early after infection, roughly equivalent numbers of WT and Fabp4/5 dKO CD8⁺ effector T cells (T_{eff}) were found in skin (FIG. 2C). Compared to skin T_{RM} isolated at day 30, skin infiltrating T_{eff} isolated at days 10 and 15 displayed a lower OCR but a higher basal extracellular acidification rate (ECAR), which corresponds to glycolysis²¹ (FIG. 11D). Deficiency in Fabp4/5 decreased the OCR of T_{RM} at day 30 post infection but had no effect on the ECAR of skin infiltrating T_{eff} (FIG. 11D). These data suggest that early skin infiltrating T_{eff} utilize glycolysis, which is unaffected by Fabp4/5 gene expression.

To establish the contribution of FABP4 and FABP5 in non-transgenic CD8⁺ T_{RM}, bone marrow chimeric mice containing a 1:1 ratio of Thy1.1⁺ CD45.1⁺ WT and Thy1.1⁺ CD45.2⁺ Fabp4/5 dKO bone marrow cells were infected with rVACV by skin scarification. At 45 days later, infected skin tissue was harvested and the frequency of VACV-pentamer⁺ CD8⁺ T cells³ was analyzed by flow cytometry. Consistent with data from OT-I experiments, fewer Fabp4/5 dKO T_{RM} were detected compared to WT T_{RM} (FIG. 211).

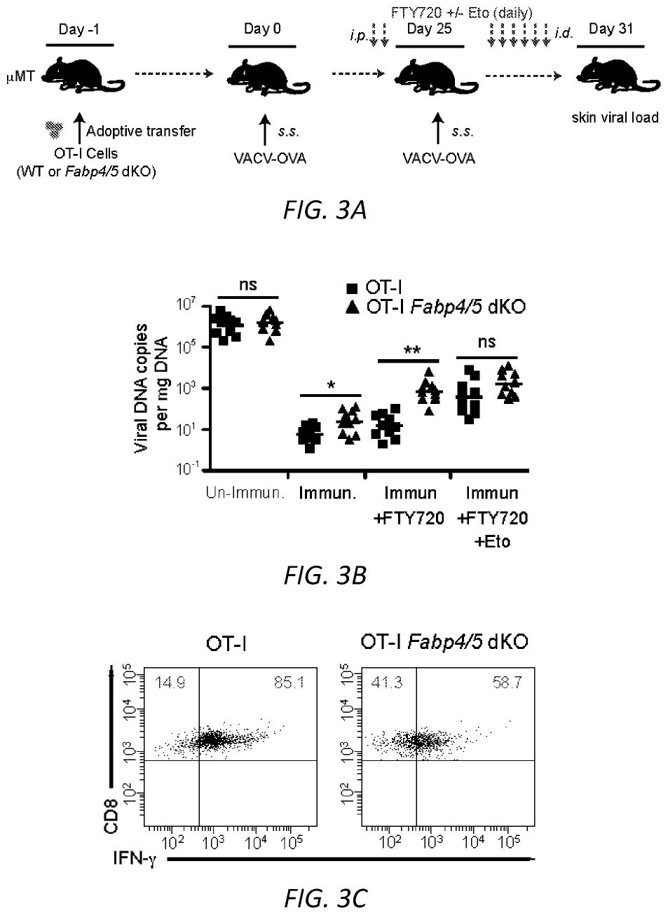

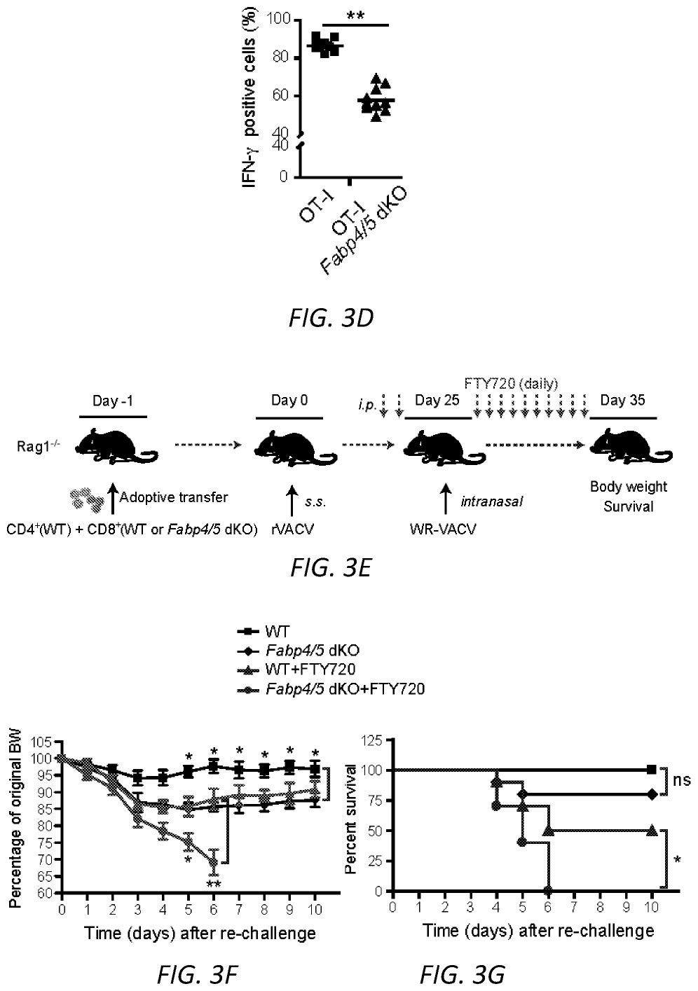

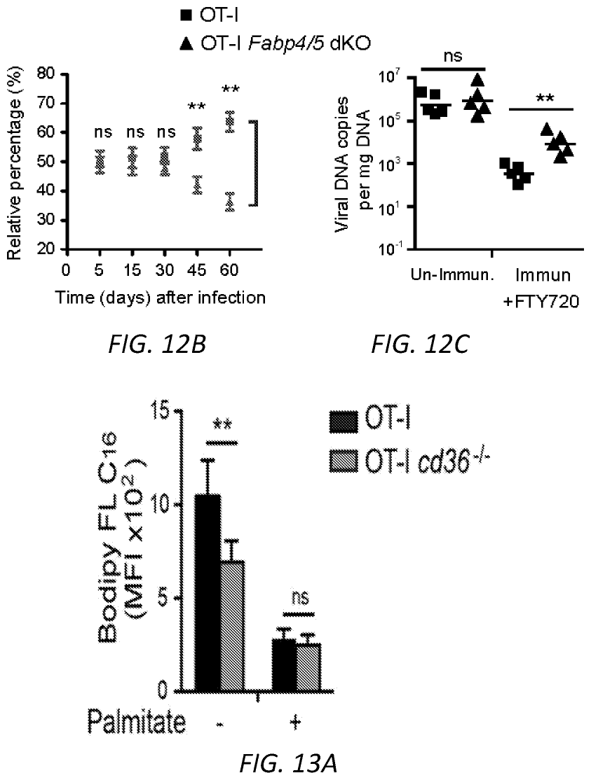

T_{RM} are more effective than are T_{CM} at clearing tissue VACV infections³. We evaluated the contribution of FABP4 and FABP5 to viral clearance of CD8⁺ T_{RM}. Mice were adoptively transferred with OT-I WT or OT-I Fabp4/5 dKO cells and then infected by skin scarification with VACV_{OVA}. Mice were re-challenged with VACV_{OVA} 25 days later and skin viral load was measured six days later (FIG. 3A). FTY720, a sphingosine-1-phosphate receptor antagonist, was injected into mice to assess the contribution of circulating T_{CM} to viral clearance (FIG. 12A). Established OT-I WT T_{RM} were highly effective at clearing virus from skin, which was rapid and unaffected by FTY720 treatment (FIG. 3B). In contrast, OT-I T_{RM} lacking Fabp4 and Fabp5 were less effective at viral clearance; this was exaggerated following FTY720 treatment (FIG. 3B). Treatment of mice with etomoxir reduced the VACV clearing capacity of OT-I WT T_{RM} to a level comparable to that of their Fabp4/5 dKO counterparts (FIG. 3B), suggesting dependence upon oxidative metabolism of FFA. Upon stimulation in vitro, OT-I Fabp4/5 dKO T_{RM} displayed impaired IFN-γ production compared to WT counterparts (FIG. 3C-D). Similar results were obtained for OT-I Fabp4/5 dKO T_{RM} residing at skin sites distant from the infection (FIGS. 12B-C).

We showed previously that lung CD8⁺ T_{RM} generated via skin VACV vaccination could partially protect mice against a lethal respiratory challenge with VACV⁹. We therefore investigated the role of FABP4 and FABP5 in this protective capacity of lung CD8⁺ T_{RM} generated by skin scarification. Rag1^{-/-} mice were reconstituted with transfer of CD4⁺ and CD8⁺ WT or Fabp4/5 dKO T cells one day before immunization with rVACV by skin scarification. At day 25, WT or Fabp4/5 dKO rVACV memory mice were challenged intranasally with lethal doses of highly pathogenic Western Reserve (WR)-VACV (FIG. 3E). Mice with WT CD8⁺ T_{RM} were protected from clinical illness (e.g. weight loss) (FIG. 3F). In contrast, mice with Fabp4/5 dKO CD8⁺ T_{RM} showed marked weight loss after challenge and were only partially protected from lethality (FIG. 3F-G). Treatment with FTY720 led to 100% mortality of mice containing Fabp4/5 dKO CD8⁺ T_{RM}, while those containing WT CD8⁺ T_{RM} were partially but significantly protected (FIG. 3F-G). These data suggest that lung Fabp4/5 dKO CD8⁺ T_{RM} were less protective against lethal respiratory VACV infection, and required the recruitment of circulating T_{CM}. In contrast, WT CD8⁺ T_{RM} alone protected 50% of mice from this overwhelming lethal VACV infection, consistent with our previous data⁹.

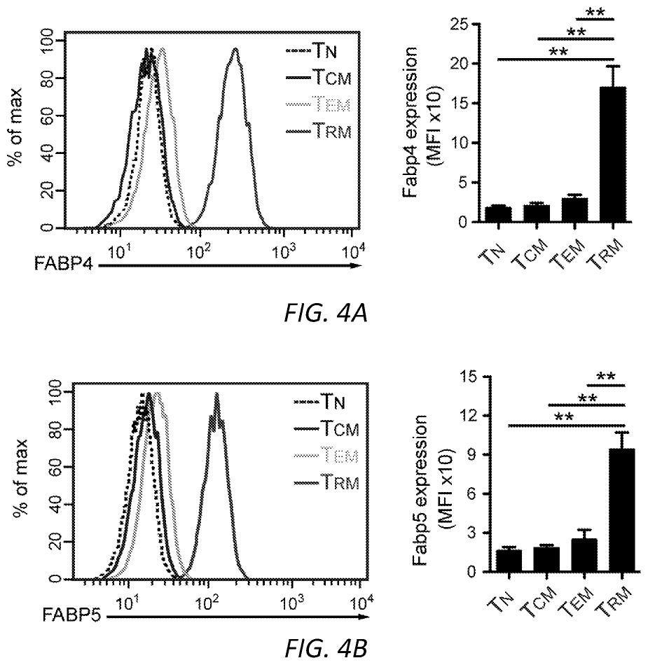

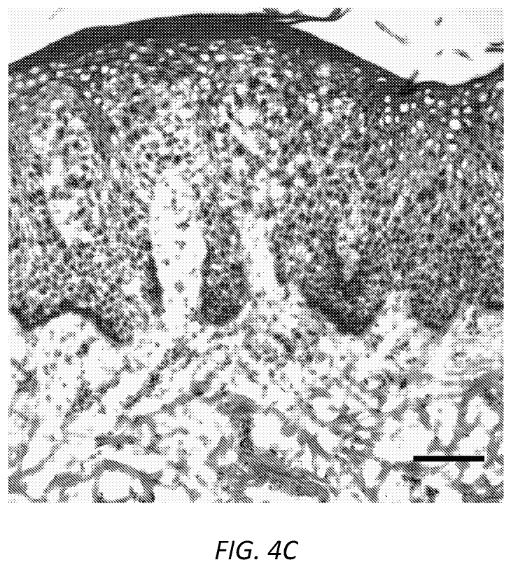

T_{RM} in human skin have been implicated in the pathogenesis of several human skin diseases, including psoriasis^{10,24,25}. We found that FABP4 and FABP5 were both strongly expressed in human skin CD8⁺ T_{RM} compared to human blood T_{N}, T_{CM} and T_{EM} by FACS analysis (FIG. 4A-B). Psoriasis is a chronic and recurring autoimmune disease (FIG. 4C) thought to be mediated by CD8⁺ T_{RM} cells^{5,26}. IF staining of lesional psoriatic skin showed a co-expression of CD8 and CD69⁵, indicating that CD8⁺ T cells in psoriasis tissue display a T_{RM} phenotype. Lipids could be visualized in lesional scalp skin and readily detectable FABP4 and FABP5 protein expression could be detected in human psoriatic skin CD8⁺ T_{RM}. Incubation in vitro with exogenous Bodipy C16 showed that human skin CD8⁺ T_{RM} internalized more exogenous FFA compared to blood T_{N}, T_{CM} and T_{EM} (FIG. 4D), suggesting a similar role of FABP4 and FABP5 in fatty acid uptake of human CD8⁺ T_{RM} as we demonstrated in mice.

Skin and other epithelial tissues are lipid-rich but nutrient-poor microenvironments^{15,27}, and CD8⁺ T_{RM} appear to utilize Mitochondrial® oxidation of exogenous FFA or other lipids to support both their longevity and protective function. Although T_{CM} depend in part on FAO for cellular metabolism^{17,28}, our data show that T_{CM} cannot effectively internalize exogenous FFA. Cell-intrinsic lipolysis and increased glycerol transport are used by T_{CM} to support metabolic programming necessary for development^{17,28,29}, but the dependence upon exogenous FFA uptake and metabolism for long-term survival is unique to T_{RM}. Additionally, it is noteworthy that similar results were obtained from mice injected intradermally with etomoxir and mice with Cpt1a knockdown in OT-I cells (FIG. 2G, FIGS. 11A-B), suggesting that the etomoxir effects on CD8⁺ T_{RM} persistence were mediated through CPT1A³⁰. Given that generation of long-lived T_{RM} are a goal of effective vaccination⁴, and that dysfunction of T_{RM} underlies many auto-inflammatory tissue disorders^{4,5}, a more detailed understanding of the unique lipid metabolic programs intrinsic to T_{RM} and how these programs might be manipulated to increase or decrease T_{RM} longevity and function, will be a subject of future investigation.

### Example 2. CD36 Deficiency Decreases FFA Uptake and T_{RM} Survival in Skin

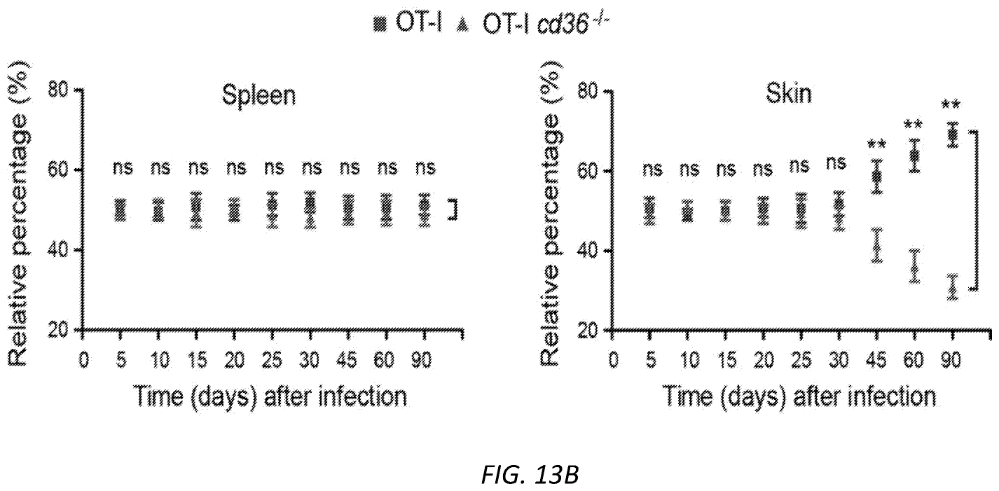

OT-1 skin T_{RM} were compared to Cd36−/− OT-1 skin TRM at day 30 in the uptaking of extracellular Bodipy conjugated palmitate. As shown from FIG. 13A, Cd36−/− TRM internalize BiodipyFL16 less efficiently than do WT OT-1 TRM. This uptake is specific, and could be blocked with unlabeled palmitate (FIG. 13A). We next performed experiments mixing equal numbers of WT OT-1 cells and cd36−/− OT-1 cells, transferring into naïve recipient mice, and then infecting the mouse with VACVOVA. TCM from spleen and T cells from skin were harvested at various time points, and the ratio of WT to cd36−/− OT-1 cells was assessed by flow cytometry. No difference could be observed between cd36−/− and WT TCM over the 90 days of the experiment (FIG. 13B). However, in skin beginning at day 45, there was a survival disadvantage of cd36−/− TRM, which gradually increased through day 90 (FIG. 13B).

### Example 3. Deficiency of Cd36 and Fabp4/5 Further Decreases FFA Uptake

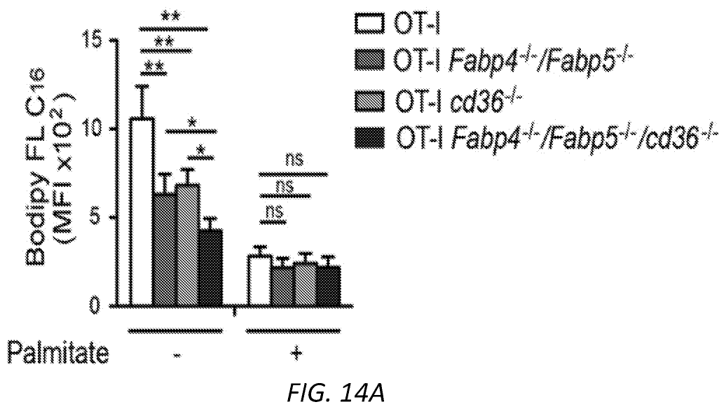

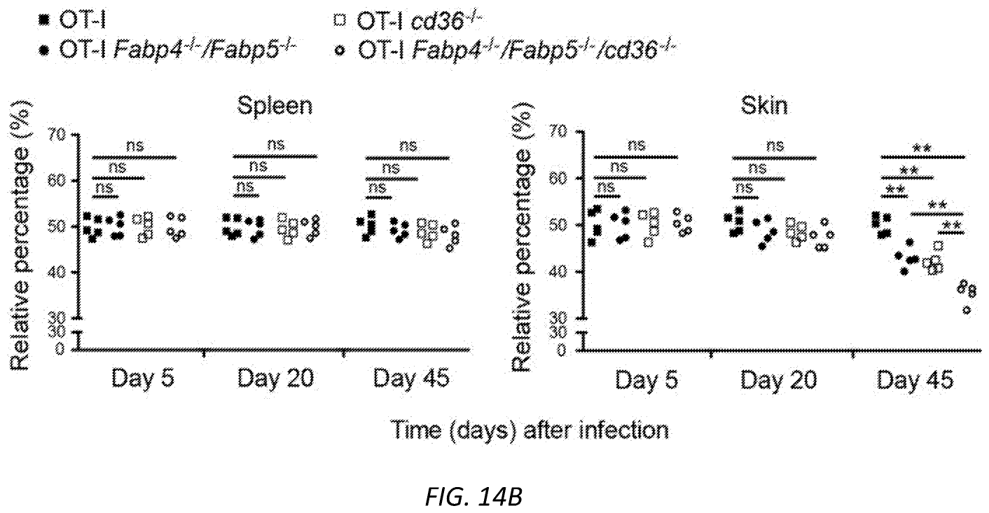

The results showed that Fabp4−/−/Fabp5−/− cd36−/− TRM cells internalize the least amount of BiodipyFL16 than do Fabp4−/−/Fabp5−/− or cd36−/− TRM cells (FIG. 14A), indicating that the effect of cd36 and Fabp4−/−/Fabp5−/− are at least additive. Next we assessed the contribution of CD36, Fabp4 and Fabp5 in the survival and maintenance of skin TRM. 50:50 congenic mixtures of Fabp4−/−/Fabp5−/− OT-1 cells and WT OT-I cells, cd36−/− OT-1 cells and WT OT-1 cells, and Fabp4−/−/Fabp5−/− cd36−/− OT-1 cells and WT OT-1 cells were adoptively transferred at day-1 and mice were skin immunized with VACVOVA. Splenic OT-1 TCM showed equal numbers of WT and gene deficient OT-1 cells, while Fabp4−/−/Fabp5−/−, cd36−/−, and Fabp4−/−/Fabp5−/− were equivalent in number to skin OT-1 cells at day 5 and 20, but showed decreased relative survival in skin at day 45 (TRM). Triple gene deficient OT-1 cells performed worst at this time point (FIG. 14B).

### Example 4. Etomoxir and Trimetazidine Block TRM Mediated Contact Hypersensitivity (CHS)

C57BL/6 mice were sensitized by topical applications of 20 μl of 0.25% DNFB diluted in acetone olive oil (3:1 v/v) at the indicated time points (day 0, 1 and 7). Challenge was performed at the infection site with 20 μl of DNFB-acetone olive oil (aOO) (0.25%), with or without etomoxir (1 ug/site) and trimetazidine (10 ug/site), at the indicated time points. Then the ear thickness of the mice was measured with a digital thickness gauge (Mitsutoyo) for the following days.

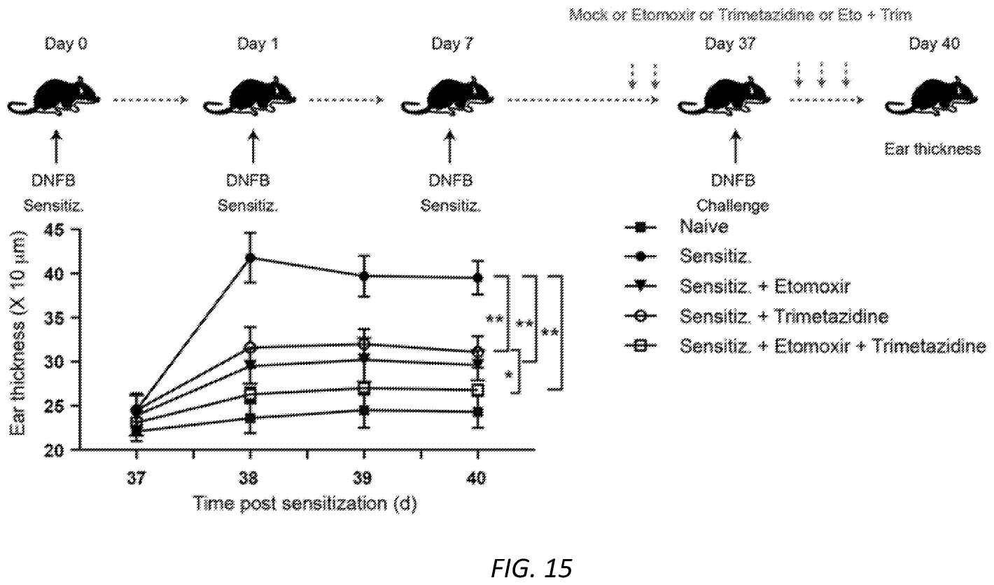

The results (see FIG. 15) showed that FAO inhibition inhibited contact hypersensitivity (CHS) reaction post DNFB re-exposure, indicating that TRM depend on exogenous FFA for survival and function, and do not persist in tissues if they cannot utilize this energy source. Various compounds that target different steps of this mechanism (FFA utilization, metabolism, and transport) are capable of depleting already established TRM in vivo.

### REFERENCES

1 Gebhardt, T. et al. Memory T cells in nonlymphoid tissue that provide enhanced local immunity during infection with herpes simplex virus. Nature immunology 10, 524-530, doi:10.1038/ni.1718 (2009).
2 Masopust, D. et al. Dynamic T cell migration program provides resident memory within intestinal epithelium. The Journal of experimental medicine 207, 553-564, doi:10.1084/jem.20090858 (2010).
3 Jiang, X. et al. Skin infection generates non-migratory memory CD8+T(RM) cells providing global skin immunity. Nature 483, 227-231, doi:10.1038/nature10851 (2012).
4 Park, C. O. & Kupper, T. S. The emerging role of resident memory T cells in protective immunity and inflammatory disease. Nature medicine 21, 688-697, doi:10.1038/nm.3883 (2015).
5 Clark, R. A. Resident memory T cells in human health and disease. Science translational medicine 7, 269rv261, doi:10.1126/scitranslmed.3010641 (2015).
6 Ariotti, S. et al. T cell memory. Skin-resident memory CD8(+) T cells trigger a state of tissue-wide pathogen alert. Science 346, 101-105, doi:10.1126/science.1254803 (2014).
7 Iijima, N. & Iwasaki, A. T cell memory. A local macrophage chemokine network sustains protective tissue-resident memory CD4 T cells. Science 346, 93-98, doi:10.1126/science.1257530 (2014).
8 Schenkel, J. M. et al. T cell memory. Resident memory CD8 T cells trigger protective innate and adaptive immune responses. Science 346, 98-101, doi:10.1126/science.1254536 (2014).
9 Liu, L. et al. Epidermal injury and infection during poxvirus immunization is crucial for the generation of highly protective T cell-mediated immunity. Nature medicine 16, 224-227, doi:10.1038/nm.2078 (2010).
10 Gaide, O. et al. Common clonal origin of central and resident memory T cells following skin immunization. Nature medicine 21, 647-653, doi:10.1038/nm.3860 (2015).
11 Mackay, L. K. et al. The developmental pathway for CD103(+)CD8+ tissue-resident memory T cells of skin. Nature immunology 14, 1294-1301, doi:10.1038/ni.2744 (2013).
12 Mackay, L. K. et al. T-box Transcription Factors Combine with the Cytokines TGF-beta and IL-15 to Control Tissue-Resident Memory T Cell Fate. Immunity 43, 1101-1111, doi:10.1016/j.immuni.2015.11.008 (2015).
13 Skon, C. N. et al. Transcriptional downregulation of Slprl is required for the establishment of resident memory CD8+ T cells. Nature immunology 14, 1285-1293, doi:10.1038/ni.2745 (2013).
14 Sanz, P. & Moss, B. Identification of a transcription factor, encoded by two vaccinia virus early genes, that regulates the intermediate stage of viral gene expression. Proceedings of the National Academy of Sciences of the United States of America 96, 2692-2697 (1999).
15 Khnykin, D., Miner, J. H. & Jahnsen, F. Role of fatty acid transporters in epidermis: Implications for health and disease. Dermatoendocrinol 3, 53-61, doi:10.4161/derm.3.2.14816 (2011).
16 Rogue, A., Spire, C., Brun, M., Claude, N. & Guillouzo, A. Gene Expression Changes Induced by PPAR Gamma Agonists in Animal and Human Liver. PPAR Res 2010, 325183, doi:10.1155/2010/325183 (2010).
17 Pearce, E. L. et al. Enhancing CD8 T-cell memory by modulating fatty acid metabolism. Nature 460, 103-107, doi:10.1038/nature08097 (2009).
18 Pollizzi, K. N. & Powell, J. D. Integrating canonical and metabolic signalling programmes in the regulation of T cell responses. Nature reviews. Immunology 14, 435-446, doi:10.1038/nri3701 (2014).
19 Hotamisligil, G. S. & Bernlohr, D. A. Metabolic functions of FABPs—mechanisms and therapeutic implications. Nat Rev Endocrinol 11, 592-605, doi:10.1038/nrendo.2015.122 (2015).
20 Maeda, K. et al. Role of the fatty acid binding protein mall in obesity and insulin resistance. Diabetes 52, 300-307 (2003).
21 van der Windt, G. J., Chang, C. H. & Pearce, E. L. Measuring Bioenergetics in T Cells Using a Seahorse Extracellular Flux Analyzer. Curr Protoc Immunol 113, 3 16B 11-13 16B 14, doi:10.1002/0471142735.im0316bs113 (2016).
22 Pike, L. S., Smift, A. L., Croteau, N. J., Ferrick, D. A. & Wu, M. Inhibition of fatty acid oxidation by etomoxir impairs NADPH production and increases reactive oxygen species resulting in ATP depletion and cell death in human glioblastoma cells. Biochim Biophys Acta 1807, 726-734 (2011).
23 Kantor, P. F., Lucien, A., Kozak, R. & Lopaschuk, G. D. The antianginal drug trimetazidine shifts cardiac energy metabolism from fatty acid oxidation to glucose oxidation by inhibiting mitochondrial long-chain 3-ketoacyl coenzyme A thiolase. Circ Res 86, 580-588 (2000).
24 Clark, R. A. et al. The vast majority of CLA+ T cells are resident in normal skin. Journal of immunology 176, 4431-4439 (2006).
25 Adachi, T. et al. Hair follicle-derived IL-7 and IL-15 mediate skin-resident memory T cell homeostasis and lymphoma. Nature medicine 21, 1272-1279, doi:10.1038/nm.3962 (2015).
26 Cheuk, S. et al. Epidermal Th22 and Tc17 cells form a localized disease memory in clinically healed psoriasis. Journal of immunology 192, 3111-3120, doi:10.4049/j.immunol.1302313 (2014).
27 Zhang, Y. et al. Epidermal Fatty Acid binding protein promotes skin inflammation induced by high-fat diet. Immunity 42, 953-964, doi:10.1016/j.immuni.2015.04.016 (2015).
28 O'Sullivan, D. et al. Memory CD8(+) T cells use cell-intrinsic lipolysis to support the metabolic programming necessary for development. Immunity 41, 75-88, doi:10.1016/j.immuni.2014.06.005 (2014).
29 Cui, G. et al. IL-7-Induced Glycerol Transport and TAG Synthesis Promotes Memory CD8+ T Cell Longevity. Cell 161, 750-761, doi:10.1016/j.cell.2015.03.021 (2015).
30 Nomura, M. et al. Fatty acid oxidation in macrophage polarization. Nature immunology 17, 216-217, doi:10.1038/ni.3366 (2016).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating or reducing risk of development or progression of an immune or inflammatory disease, the method comprising administering a therapeutically effective amount of (i) one or more inhibitors of mitochondrial exogenous lipid or free fatty uptake and/or (ii) one or more inhibitors of mitochondrial fatty acid oxidation or metabolism to a subject in need thereof.

2. The method of claim 1, wherein the disease is mediated by resident memory T cells (T_{RM}).

3. The method of claim 2, wherein the T_{RM}-mediated disease is an autoimmune or auto-inflammatory disease or disorder involving non-lymphoid tissue.

4. The method of claim 3, wherein the autoimmune or auto-inflammatory disease or disorder is selected from one or more of the following: (a) diseases of the skin; (b) diseases of the gastrointestinal (GI) Tract; (c) endocrine or metabolic diseases (d) diseases of the lung; (e) diseases of the bones or joints or (f) diseases of the CNS.

5. The method of claim 4, wherein the disease of the skin is selected from (a) psoriasis; (b) vitiligo; (c) graft vs host disease; (d) contact dermatitis; (e) alopecia areata; or (f) eczematous dermatitis.

6. The method of claim 4, wherein the disease of the GI tract is Crohn's Disease, irritable bowel disease, or ulcerative colitis.

7. The method of claim 4, wherein the disease of the lung is asthma.

8. The method of claim 4, wherein the endocrine or metabolic disease is Type I diabetes (insulin dependent diabetes mellitus).

9. The method of claim 4, wherein the disease of the bones or joints is rheumatoid arthritis or a spondylarthropathy.

10. The method of claim 4, wherein the disease of the CNS is multiple sclerosis.

11. The method of claim 1, wherein the one or more inhibitors of mitochondrial exogenous lipid or free fatty uptake comprise an inhibitor or antagonist of CD36 or a Fatty Acid Binding Protein (FABP).

12. The method of claim 11, wherein the inhibitor or antagonist of FABP is an inhibitor or antagonist of FABP4 or FABP5.

13. The method of claim 12, wherein the inhibitor or antagonist of FABP4 or FABP5 is carbazole butanoic acid, aryl sulfonamide, sulfonylthiophene derivative, 4-hydroxypyrimidine, tetrahydrocarbazole derivative, 2,3-dimethylindole derivative, benzoylbenzene, biphenyl-alkanoic acid derivative, 2-oxazole-alkanoic acid derivative, tetrahydropyrimidone, pyridone, pyrazinone, aryl carboxylic acid, tetrazole, triazolopyrimidinone, BMS309403; pyrazole, 4-{[2-(methoxycarbonyl)-5-(2-thienyl)-3-thienyl]amino}-4-oxo-2-butenoic acid or ((2′-(5-ethyl-3,4-diphenyl-1H-pyrazol-1-yl)(1,1′-biphenyl)-3-yl)oxy)-acetic acid; an indole derivative, triazolopyrimidinone derivative, Pyrazole; SBFI26 (alpha-2,4-diphenylcyclobutane-1,3-dicarboxylic acid mono-1-naphthyl ester) and other α-truxillic acid derivatives, e.g., SBFI50 (alpha-2,4-diphenylcyclobutane-1,3-dicarboxylic acid mono-2-naphthyl ester), SBFI60 (alpha-2,4-diphenylcyclobutane-1,3-dicarboxylic acid mono-1-naphthyl amide), and SBFI62 (2,4-diphenylcyclobutane-1,3-dicarboxylic acid di-1-naphthyl amide), or an inhibitory nucleic acid.

14. The method of claim 1, wherein the one or more inhibitors of mitochondrial fatty acid beta oxidation or metabolism is an inhibitor of carnitine palmitoyltransferase 1 (CPT1).

15. The method of claim 13 wherein the inhibitor of CPT1 is etomoxir; 2-tetradecylglycidic acid (TDGA); ST1326 (Teglicar); or perhexiline (2-(2,2-dicyclohexylethyl)piperidine) perhexiline (2-(2,2-dicyclohexylethyl)piperidine), or a derivative thereof, or an inhibitory nucleic acid.

16. The method of claim 1, further comprising administering a PPAR gamma inhibitor.

17. The method of claim 16, wherein the PPAR gamma inhibitor is GW9662, or a derivative thereof.

18. The method of claim 1, wherein the disease is a disease of the skin, and administration of the one or more inhibitors is by topical delivery.

19-36. (canceled)
